(19) United States
(12) Patent Application Publication
(10) Pub. No.: US 2023/0389677 A1
(43) Pub. Date: Dec. 7, 2023

(54) A RADIATION SHIELDING GARMENT, A WEIGHT TRANSFER SUPPORT SYSTEM, AND METHOD OF MAKING AND USING THE SAME

(71) Applicant: Salus Scientific Corp, Mount Pleasant, SC (US)

(72) Inventors: Todd FLOHR, Mount Pleasant, SC (US); Nick TOMCZAK, Richlands, NC (US); Leslie FLOWERDAY-PLANK, Wyoming, MN (US); Richard .S. MOORE, Wilmington, NC (US); Leroy TRUELOVE, Wilmington, NC (US); William BUCHANAN, Wrightsville Beach, NC (US)

(21) Appl. No.: 18/234,796

(22) Filed: Aug. 16, 2023

Related U.S. Application Data
(63) Continuation-in-part of application No. 18/134,394, filed on Apr. 13, 2023.
Application No. 18/134,394 is a continuation of application No. 16/910,864, filed on Jun. 24, 2020, now Pat. No. 11,627,795.
Application No. 16/910,864 is a continuation-in-part of application No. 15/896,235, filed on Feb. 14, 2018, now Pat. No. 10,729,195.
(60) Provisional application No. 63/415,204, filed on Oct. 11, 2022.
(60) Provisional application No. 63/394,474, filed on Aug. 2, 2022.
(60) Provisional application No. 62/458,623, filed on Feb. 14, 2017.

(51) Int. Cl. A45F 3/10; A45F 3/04; A42B 5/00; G21F 3/02

(52) U.S. Cl. CPC A45F 3/10; A45F 3/04; A42B 5/00; G21F 3/02; A45F 2003/045

(57) ABSTRACT

A shoulder transfer weight support apparatus including a hip or waist belt, a back support, and one or more shoulder extensions. The support apparatus includes an integrated radiation shielding unit. Optionally, the apparatus includes a face shield at least partially made of a radiation shielding material that is configured to releasable attach to the apparatus. The face shield can be a full face shield or a partial face shield.

### CROSS-REFERENCE TO RELATED APPLICATIONS

The present application claims the benefits of U.S. Patent Application No. 63/415,204 filed on Oct. 11, 2022, and claims the benefits of U.S. Patent Application No. 63/394,474, filed on Aug. 16, 2022, and is a Continuation-In-Part of U.S. application Ser. No. 18/134,394, filed on Apr. 13, 2023, which is a Continuation of U.S. application Ser. No. 16/910,864, filed on Jun. 24, 2020, now U.S. Pat. No. 11,627,795, which is a Continuation-in-Part of U.S. application Ser. No. 15/896,235, filed on Feb. 14, 2018, now 10,729,195, which claims the 62/458,623 filed on Feb. 14, 2017, the entire contents of each of the above-referenced applications are incorporated herein by reference in their entireties for all purposes.

### BACKGROUND OF THE INVENTION

### Field of the Invention

The present disclosure generally relates to a radiation shielding medical garment, shoulder transfer weight support system, and method of making and using the same and more particularly, to a supportive device for a radiation shielding garment, and related accessories, which are configured to be worn by medical professionals and others exposed to radiation.

### SUMMARY OF THE INVENTION

Accordingly, the invention is directed to a radiation shielding garment, a weight transfer support system, and method of making and using the same that substantially obviates one or more of the problems due to limitations and disadvantages of the related art.

An advantage of the invention is to an integrated radiation shielding garment with a weight transfer support system.

Still yet another advantage is to a light weight radiation shielding apparatus and accessories.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, a radiation shielding system including a weight supporting device and radiation shielding apparatus, e.g., radiation shielding garment. The weight supporting device includes an exoskeleton configured to bear a weight of a radiation shielding garment. The radiation shielding garment is configured to be arranged and at least partially supported by the weight supporting device and includes one or more of a vest, a skirt, and a scarf. At least a portion of the radiation shielding apparatus is configured to be supported by the weight supporting device.

In another aspect of the present invention, a method of assembling a radiation shielding apparatus including forming a laminate having an outer layer, an inner layer, and a core material configured to be arranged between the outer and inner layer. The method also includes basting the core material between the outer layer and the inner layer, and the basting includes clipping the layers together, stitching the layers together, and binding the radiation shielding vest.

This Summary section is neither intended to be, nor should be, construed as being representative of the full extent and scope of the present disclosure. Additional benefits, features and embodiments of the present disclosure are set forth in the attached figures and in the description hereinbelow, and as described by the claims. Accordingly, it should be understood that this Summary section may not contain all of the aspects and embodiments claimed herein.

Additionally, the disclosure herein is not meant to be limiting or restrictive in any manner. Moreover, the present disclosure is intended to provide an understanding to those of ordinary skill in the art of one or more representative embodiments supporting the claims. Thus, it is important that the claims be regarded as having a scope including constructions of various features of the present disclosure insofar as they do not depart from the scope of the methods and apparatuses consistent with the present disclosure (including the originally filed claims). Moreover, the present disclosure is intended to encompass and include obvious improvements and modifications of the present disclosure.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a left side perspective view of a shoulder transfer weight support system according to an embodiment of the invention;

FIG. 2 is rear right side perspective view thereof;

FIG. 3 is a back perspective view thereof;

FIG. 4 is a front perspective view thereof;

FIG. 5 is a left side perspective view thereof;

FIG. 6A is a top perspective view thereof with face shield according to another embodiment of the invention;

FIG. 6B is a right side perspective view thereof;

FIG. 6C is a front perspective view thereof;

FIG. 6D is a rear perspective view thereof;

FIG. 7A is a top perspective view thereof with a face shield according to another embodiment;

FIG. 7B is a right side perspective view thereof;

FIG. 7C is a front perspective view thereof;

FIG. 7D is a rear perspective view thereof;

FIG. 8A is a top perspective view with a face shield according to another embodiment;

FIG. 8B is a right side perspective view thereof;

FIG. 8C is a front perspective view thereof;

FIG. 9A is a top perspective view with a face shield according to another embodiment;

FIG. 9B is a right side perspective view thereof;

FIG. 9C is a front perspective view thereof;

FIG. 10A is a right side front perspective view of a shoulder transfer weight support system according to another embodiment of the invention;

FIG. 10B is a right side back perspective view thereof;

FIG. 10C is a back perspective view thereof;

FIG. 10D is a magnified view of portion A′ of FIG. 10C view thereof;

FIG. 10E is a partially disassembled view of FIG. 10D view thereof;

FIG. 10F is a magnified view of portion A′ of FIG. 10C view thereof;

FIG. 10G is a partially disassembled view of FIG. 10F view thereof;

FIG. 10H is a perspective view of shoulder extensions thereof;

FIG. 10I is a perspective view of an attachment member;

FIG. 10J is a partially disassembled perspective view of an attachment member and upper back plate thereof;

FIG. 10K is a partially disassembled perspective view of an attachment member and lower back plate thereof;

FIG. 10L is a right side front perspective view of FIG. 10A with a waist belt and padding according to another embodiment of the invention;

FIG. 11 is a front view of a radiation shielding apparatus according to an embodiment of the invention;

FIG. 12A is a front view of a radiation shielding vest according to an embodiment of the invention;

FIG. 12B is a back view of a radiation shielding vest according to an embodiment of the invention;

FIG. 13 is a dissembled view of the radiation shielding vest of FIGS. 11A and 11B according to an embodiment of the invention;

FIG. 14 is an illustrative view of the radiation shielding apparatus in a first orientation according to another embodiment of the invention;

FIG. 15 is an illustrative view of the radiation shielding apparatus in a second orientation;

FIG. 16 is an illustrative view of FIG. 14 in a third orientation;

FIG. 17 depicts a radiation shielding apparatus in a first orientation according to another embodiment of the invention;

FIG. 18 depicts a radiation shielding apparatus of FIG. 17 in a second orientation according to another embodiment of the invention;

FIG. 19 depicts a radiation shielding apparatus of FIG. 17 in a disassembled view;

FIG. 20 depicts a radiation shielding apparatus in a first orientation according to another embodiment of the invention;

FIG. 21 depicts a radiation shielding apparatus of FIG. 20 in a disassembled view; and

FIG. 22 illustrates a method of making a radiation apparatus according to an embodiment of the invention.

### DETAILED DESCRIPTION OF THE INVENTION

The following detailed description of the invention is directed to a radiation shielding garment, shoulder transfer weight support system, and method of making and using the same that substantially obviates one or more of the problems due to limitations and disadvantages of the related art, and is presented to enable any person skilled in the art to make and use the disclosed subject matter in the context of one or more particular implementations. Various modifications, alterations, and permutations of the disclosed implementations can be made and will be readily apparent to those skilled in the art, and the general principles defined may be applied to other implementations and applications, without departing from scope of the disclosure. The present disclosure is not intended to be limited to the described or illustrated implementations, but to be accorded the widest scope consistent with the described principles and features.

Appearances of the phrases “an embodiment,” “implementation,” “an example,” or similar language in this specification may, but do not necessarily, refer to the same embodiment, to different embodiments, or to one or more of the figures. The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps unless explicitly stated otherwise.

In order to more fully appreciate the present disclosure and to provide additional related features, each of the following references 1-15 are fully incorporated herein by reference in their entireties as fully set forth herein and also incorporated by reference for the specific teachings as follows:
(1) U.S. Pat. No. 4,286,170 issued to Moti, which relates to a protective face mask designed to shield an x-ray technician or machine operator primarily from random secondary or scatter x-rays deflected toward his face, head and neck by the table, walls, equipment and other reflecting elements in an x-ray room or chamber, during the period of exposure while adjacent the object or person being exposed to the x-ray beam. Moti is also directed towards a face mask and chest shield device which can be mounted on a patient's shoulders in reverse attitude to protect the back of a patient's head and neck from the x-ray beam while being exposed to such beams for chest or upper body portion study and examination. The face mask is relatively or substantially transparent and contains lead in combination with a plastic ionomer or comonomer, which absorbs or resists penetration, to a degree, of the random deflected secondary or scatter x-rays or the x-ray beam through the mask. The face mask is removably attachable to the chest shield for facile application of the device to and support upon the shoulders of the technician or the patient.
(2) U.S. Pat. No. 4,386,277 issued to Forshee, which relates to a protective face mask to shield an x-ray technician or machine operator primarily from random secondary or scatter x-rays deflected toward his face, head and neck by the table, walls, equipment and other reflecting elements in an x-ray room, during the period of exposure while adjacent the object or person being exposed to the x-ray beam. The face mask is formed of a lens portion supported in a plastic frame, upon the lower portion of which is removably attached a bib shield that in turn is removably attachable to an apron. The frame of the face mask, bib shield and apron are preferably lined with lead sheets to protect the neck, face, chest and body of the technician from the random secondary or scatter x-ray beams. The face mask and bib shield can be formed of a molded plastic material as a unitary device, the lens portion being attached to the frame therefor.
(3) U.S. Pat. No. 4,859,184 issued to Hazard, which relates to a face shield device for protecting the wearer's eyes and face from debris, bacteria, and the like, includes an arcuate transparent face shielding panel and a support for holding the face shielding panel in front of and spaced outwardly away from the wearer's face. The support is attached to the face shielding panel and lays on the wearer's chest. In one embodiment, the support is suspended from the wearer's neck by a flexible band which fits around the wearer's neck. In another embodiment, the support is suspended from the wearer's shoulders by shoulder engaging hook members. The face shield device may also include means to protect the wearer from ultra-violet radiation or other types of radiation harmful to the face and eyes.
(4) U.S. Pat. No. 5,022,099 issued to Walton, which relates to an improved radiation helmet having a radiation opaque liner and a replaceable hygienic tissue that is worn over the head of a dental patient during X-rays to protect the head from stray radiation. An alternative construction is to use radiation opaque materials for the radiation helmet and to omit the liner.
(5) U.S. Pat. No. 5,038,047 issued to Still, which relates to a lead impregnated shielding material hood to protect the head, brain, hypothalamus, and the master glands including a radiation protective shield for preventing direct impingement of radiation rays on the brain, neck, some salivary glands, thyroid and the adjacent body areas of head and eyes and adjacent tissues, of the patient being x-rayed. Having extraoral portion being of shape and size to cover the lead impregnated shielding material, and a substantial area of the extraoral anatomy of the head of the person to thereby intercept rays and prevent direct and/or stray penetration impingement thereon. Having physically protection of the parotid, sublingual and salivary glands. A radiation protective lead impregnated shielding hood shield being to prevent head and eyes from contact with x-rays or secondary x-rays which involve substantially large or intense radiation treatments.
(6) U.S. Pat. No. 5,499,965 issued to Sanchez, which relates to a shaped lifting belt and method in which the shape is provided by deepening the central lumbar panel in the rear to a depth of at least an additional half of the depth of the side wraps, providing the lumbar panel with expandable elements, and then separating the ends of the wrap from the lumbar panel by shape panels which are stretchable and positioned to engage the upper rear portion of the hips. The method of the invention requires the wearer to first engage the lifting belt assembly and secure the suspenders in the forward desirable position. Thereafter, the shape straps are secured to the inner portion of the wrap ends. The wrap ends are then secured to a central portion of the body followed by substantially encircling the same with the side pulls. Where coloring is desired, the suspender assembly may be of a different color than the belt itself, and optionally a removable side pull may be color coded or otherwise decorated to suit the application.
(7) U.S. Pat. No. 5,500,954 issued to Smith, et al., which relates to a safety system shield which protects the head, facial, throat, and upper thorax regions of the body from exposure to biological, chemical, and physical matter that may take the form of solids, liquids, or aerosols, and includes a thin flexible shield which is curved and transparent. It is shaped to rest on the anterior shoulder and thorax regions of the body, thereby allowing the head to look in any direction. This relieves the wearer's head of the weight of the shield while affording maximum protection. The safety shield system is ultra-lightweight, thus reducing the wearer's fatigue, and eliminates headaches, resulting in the enhancement of safety and the reduction of accidents. The safety shield is flexible allowing it to conform to the size and curvature of the wearer.
(8) U.S. Pat. No. 8,933,426 issued to Rees, which relates to a personal radiation protection system that substantially contours to an operator's body is suspended from a suspension means. The garment is operable to protect the operator from radiation. The suspension means is operable to provide constant force and allows the operator to move freely in the X, Y and Z planes simultaneously, such that the protective garment, face shield, illumination means or other attachments integrated to the system are substantially weightless to the operator. A face shield and arm cover can also be incorporated with the system, such that the face shield and arm cover are substantially weightless to the operator. The suspension means may be mounted to the ceiling, a vertical wall, the floor, or on a mobile platform.
(9) U.S. Pat. No. 9,504,307 issued to Burnett, et al., which relates to a flexible load bearing system includes a plurality of vertebrae. Each vertebra has a first portion, a second portion, and a socket formed in it. A plurality of links each has a ball at a first end and at a second end. At least one of the plurality of balls is disposed in one of the plurality of sockets, thus forming a column. A tension mechanism is mated to the first portion and to the second portion and configured to pull or push the first portion and the second portion together or apart, respectively.
(10) U.S. Pat. No. 9,629,443 issued to Searle, et al., which relates to a load carriage frame that includes a shoulder yoke, and a belt separated from the yoke by a multi-element connecting brace. The brace is telescopic in nature in that a flexible but rigid tongue, extending from a first section, is permitted to slide within a guide on a second section, thereby allowing the length of the connecting brace to be altered. The multi-element connecting brace is centrally coupled to the yoke and centrally coupled to the belt and configured to act as a force path by transferring weight that, in use, is loaded onto or through the yoke and into the belt thereby effecting selected weight re-distribution onto the pelvic girdle of a wearer about which the belt, in use, is secured. Additionally, a quick release mechanism may further alter the length of the connecting brace by allowing the position of a rotational coupling point to be altered relative to a housing of the quick release mechanism. The quick release mechanism can, in fact, be used with a single piece connecting brace.
(11) U.S. Pat. No. 10,016,251 issued to Czajka, et al., which relates to a shield is disclosed for protecting a user from radiation emitted during X-ray imaging. The shield includes a lead layer for absorbing the radiation. The shield comprises first and second opposing flaps configured to position a first portion of the shield around an upper part of the user's head in a deployed configuration, whereby the portion, a component having first and second ends that are attached to the first and second opposing flaps, respectively, wherein the component is adapted to expand to enable the first portion of the shield to adjust to fit around the upper part of the user's head, and third and fourth opposing flaps are configured to position a second portion of the shield around a lower part of the user's head.
(12) U.S. Pat. No. 10,448,685 issued to Czajka, et al., which relates to a protective apparel support system is disclosed comprising a support frame configured to rest on the shoulders of a wearer, the support having a first shoulder member, a second shoulder member and a shield engagement portion. A shield is selectively couplable to the support and protective apparel is coupled to the shield.
(13) U.S. Pat. No. 10,729,195, which is directed towards a wearable weight support that transfers the weight of heavy garments that rely on shoulder support off of the shoulders to the hips of the user. The device provides additional posterior radiation protection to the wearer as the back support can be made from radio-opaque materials. The device also provides lumbar support and results in less body heat containment through natural venting. The device is easily adjustable to any body type and allows the wearer to move more freely in their work environment. The invention can be used to transfer the weight of a backpack or other systems which rely on shoulder support to function.
(14) U.S. Patent Appl. Publication No. 2011/0174997 issued to Rees, which relates to an improved personal radiation protection system that substantially contours to an operator's body and is suspended from a suspension means. The garment is operable to protect the operator from radiation. The suspension means is operable to provide constant force and allows the operator to move freely in the X, Y and Z planes simultaneously, such that the protective garment, face shield, or other attachments integrated into the system are substantially weightless to the operator. The suspension means may be mounted to the ceiling, a vertical wall, the floor, or on a mobile platform. The suspension means may comprise an articulating arm, a balance arm, or a manipulator, and the radiation protection system is suspended generally about its center of gravity.
(15) U.S. Pat. Publ. No. 2020/0315325, which is directed towards a shoulder transfer weight support generally having a hip or waist belt, a back support, and one or more shoulder extensions. The back support has an upper back plate attached to the shoulder extensions and a lower back plate attached to one or more hip belts. The lower back plate features a bracket shaped to receive an end of the attachment bar that joins it to the upper back plate. A spring-loaded plunger can be inserted into the attachment bar to allow for height adjustment of the device. A face shield is at least partially made of a radio-opaque material and can attach to the back support. One attachment option is a yoke mount with vertical sections at either end of the yoke mount. A second option involves a mount that attaches to the shoulder extension(s). The device can use a full-face shield or a face shield with an open front.

In one embodiment, a radiation shielding apparatus or radiation shielding garment is integrated or not integrated with a shoulder transfer weight support system as described herein. When integrated the radiation shielding garment is part of the weight transfer apparatus. The term radiation shielding apparatus and radiation shielding garment are used interchangeably herein. The term radiation is radiation that is blocked and/or attenuated by the radiation apparatus and can include either or both primary and scattered radiation. Primary radiation is radiation coming directly from a source, such as a radioactive substance or an x-ray tube, without interactions with matter. Scattered radiation includes radiation that spreads out in different directions from a radiation beam when the beam interacts with a substance, such as body tissue.

In one embodiment, the radiation shielding garment includes a vest garment, a skirt garment, and a scarf garment. The scarf garment may be also referred to as a thyroid shield. The radiation shielding garment includes radiation shielding materials, e.g., lead material, non-lead material and/or low lead material. The radiation shielding materials are configured to protect against radiation, e.g., against the highest attenuation levels available, for health professionals in various environments, e.g., Cath labs, Radiology rooms, health imaging departments and the like. In one embodiment, the radiation shielding material is described with reference to U.S. Provisional Appl. No. 63/398,474, which is incorporated herein by reference for those teachings. The radiation shielding material can include materials that are flexible and lightweight such as flexible lead and lead-free vinyl radiation shielding materials from Kemmetech. In one embodiment, the lead equivalent values of one or more of 0.125 mm, 0.167 mm, 0.175 mm, 0.25 mm, 0.35 mm, 0.50 mm, 0.70 mm, and 1.00 mm depending on level of protection desired. The composition can include emulsion polymers, plasticizers, finely divided lead and lead-free particles, stabilizers and pigments.

In one embodiment, the core material includes one or more of a light lead radiation shielding material, a lead radiation shielding material, and a lead free radiation shielding material configured to a have a lead equivalence in range from about 0.125 mm to about 1.0 mm or greater.

In one embodiment, the radiation shielding material is configured to a have a lead equivalence in range from about 0.125 mm to about 1.0 mm.

In a preferred embodiment, the radiation shielding material includes a radiation protection vinyl, e.g., one or more sheets of material, constructed of a high atomic weight element on one layer and a lower atomic weight element on another layer. In another embodiment, the material is edge bilayer from Kemmetech having a construction of two distinct homogenous layers as one integral sheet. The material can be distributed evenly to as specially graded metal particles (e.g., lead/non-Lead) within the vinyl matrix of each layer and therefore a consistent level of protection. In one embodiment, one or more of the following can be used Kemmetech LE Edge Bilayer 0.175 Bilayer Low Lead, 0.25 LE Bi-Layer Low Lead, 0.35 LE Bi-Layer Low Lead, 0.5 LE Bi-Layer Low Lead, 0.175 0.25 LE Bi-Layer Lead Free, 0.250 LE Bi-Layer Lead Free, 0.35 LE Bi-Layer Lead Free, 0.50 LE Bi-Layer Lead Free, 0.125 LE Lightweight Lead, 0.167 LE Lightweight Lead, 0.175 LE Lightweight Lead, 0.250 LE Lightweight Lead, 0.350 LE Lightweight Lead, 0.125 LE Superlight Lead, 0.167 LE Superlight Lead, 0.175 LE Superlight Lead, 0.250 LE Superlight Lead, 0.350 LE Superlight Lead, 0.125 LE Lead Free, 0.167 LE Lead Free, 0.175 LE Lead Free, 0.250 LE Lead Free, 0.350 LE Lead Free combinations of the same and the like.

In another embodiment, other lead-free and lead composite products, e.g., containing low Z (atomic number) materials either exclusively or in a mixed metal composite can be utilized.

In one embodiment, a shoulder transfer weight support system is configured to be used with a radiation shielding garment. The weight supporting device includes an exoskeleton configured to bear the weight of a radiation shielding garment. In various embodiments, the radiation shielding garment is configured to be arranged and at least partially supported by the weight supporting device. In another embodiment, the radiation shielding apparatus includes a vest garment, a skirt garment, and a scarf garment, wherein the vest garment, the skirt garment, and the scarf garment are configured to be at least partially supported by the weight supporting device and also configured to protect the user from radiation exposure.

In one embodiment, a method of assembling a radiation shielding vest includes forming a laminated structure, e.g., having an outer layer, an inner layer, and a radiation shielding material, e.g., core material, arranged between the outer and inner layer. The method includes basting the core material between the outer layer and the inner layer, wherein the basting includes attaching or clipping the layers together, e.g., by stitching the layers together or other suitable techniques, e.g., fastening, zipping, adhering, fusing, tacking, seaming, hemming. In various embodiments, the method includes binding the radiation shielding vest to form a wearable one-piece garment configured to cover a user's torso. Other stitching techniques can also be used in any of the process steps described herein for attaching the outer layer to the inner layer.

In one embodiment, the weight supporting device includes an exoskeleton configured to bear at least some of the weight of a radiation shielding garment. In various embodiments, the radiation shielding garment is configured to be arranged and at least partially supported by the weight supporting device. In another embodiment, the radiation shielding garment includes a vest that is configured to protect a user's upper body, e.g., torso, a skirt configured to protect a user's lower extremities, e.g., legs, and a scarf configured to protect a user's neck and thyroid region. The shielding garment is configured to be at least partially supported by the weight supporting device and configured to protect the user from radiation exposure. Optionally and/or alternatively, the radiation shielding apparatus can also include sleeves, pants, and or shirts constructed as described herein.

In one embodiment, the vest, skirt, and scarf may each be configured to couple to the weight supporting device, e.g., exoskeleton. The weight supporting device may include a waist portion including opposing ends coupled by a hook-and-loop fastener. The waist portion may be configured to encircle a user's waist and/or hips. To further secure the weight supporting device to a user's waist, the waist portion may also include a buckle in addition to the hook-and-loop fastener or other securing mechanism. The weight supporting device may further include one strap or more straps configured to stabilize the shoulder portion during use. The strap is also configured to adjust to users of varying sizes.

In one embodiment, the vest may include interior portions, e.g., belt loops, that may complement the shoulder and waist portions of the weight supporting device. Accordingly, a user may slide the portions of the weight supporting device, e.g., mantis bars or shoulder bars, through the interior loops, thereby integrating the weight supporting device with the vest. The weight supporting device and vest may further include a plurality of complementary attachment mechanisms, e.g., snaps, buttons, magnets, that are configured to further secure the weight supporting device to the vest.

In one embodiment, the scarf includes opposing ends configured to be at least partially coupled by a hook-and-loop fastener. In another embodiment, the scarf may include a buckle configured to couple the opposing ends of the scarf. In one exemplary embodiment, the skirt may include opposing ends configured to be at least partially coupled by a buckle. The skirt may further include a plurality of hook-and-loop fasteners configured to further secure the opposing ends to one another. In one exemplary embodiment, the vest may include a plurality of hook-and-loop fasteners configured to enable attachment of various accessories and components, including undergarments, weight supporting device components, padding, cushions, labels, and the like.

In one embodiment, the radiation shielding material or core material includes one or more of a lead material, a lead free material, a low lead material, combinations of the same and the like. In one embodiment, a lead material is utilized and the lead material is configured to attenuate, e.g., block or partially block, radiation. Other high-density materials can also be utilized as the radiation shielding material, e.g., a lead free material that exhibits high attenuation of radiation such as heavy metals, tin, antimony, tungsten, and bismuth and titanium amalgamations, lead-polyethylene-boron composites, combinations of the same and the like. In one exemplary embodiment, the core material includes 0.25 mm lead equivalence or greater. In another embodiment, the core material includes up to 0.5 mm lead equivalence or greater.

In one embodiment, the efficacy of radiation shielding garments herein can measured across a range of radiation beam qualities. For example, penetration may be measured using beams of nominal 60, 70, 80, 100, and 120 kilovoltage peak (kVp). In X-ray production, for example, penetrance is characterized by the number of photons reaching the image receptor to discern differences between structures. An adequate penetrance ensures the ability to separate definable structures of interest. In embodiments herein the radiation shielding garment protects health professionals in cath labs, radiology rooms and health imaging departments from this kind of penetration. The kVp is the difference in potential applied to an X-ray tube and is directly proportional to the average energy of the X-ray spectrum produced, referred to as X-ray quality. The kVp plays a role in adjusting the amount of penetration and exposure in an acquisition.

In one embodiment, a method of making a radiation shielding vest includes forming a laminated structure. The laminated structure includes an outer layer, an inner layer, and a core material arranged between the outer layer and inner layer. The method further includes attaching the core material between the outer layer and the inner layer. In a preferred embodiment, the core material is attached with one or more basting stitches. A basting stich is a long, loose stitch designed to hold fabrics together temporarily, but is intended to be removed. For example, the side seams of a skirt are basted together to check the fit, then the final seam is sewn, and the basting stitch is removed. In one embodiment, the core material between the outer layer and the inner layer is attached by attaching or clipping the outer and inner layers together, e.g., by stitching the layers together or other suitable techniques, e.g., fastening, zipping, adhering, fusing, tacking, seaming, or hemming to form a wearable one-piece garment configured to cover a user's torso.

In one embodiment, the outer layer is made of vinyl, nylon, polyester, polypropylene, rubber, cotton, an elastane mixture, a cotton-polyester combination, and/or the like. In one embodiment, the inner layer is made of vinyl, nylon, polyester, polypropylene, rubber, cotton, an elastane mixture, a cotton-polyester combination, and/or the like. In one embodiment, the core material includes radiation shielding material as described herein.

In one embodiment, the method of forming the radiation shielding garment includes forming a garment, e.g., vest, skirt, scarf. The method includes laminating multiple layers around a radiation shielding material layer. The radiation shielding material layer may include a laminated layer of radiation shielding material. The method includes cutting a first inner layer, a second inner layer, and a third inner layer to a desired shape or pattern. The method includes attaching the layers together. In one embodiment, attaching the layers can be done with a plurality of attachment mechanism, e.g., snaps, hook and loop fasteners, glue, and the like. In addition, at least a portion of the radiation shielding garment is configured to be attached to at least a portion of the weight transfer support system. In one embodiment, the inner layer of the garment includes a plurality of attachment mechanisms, e.g., snaps, hook and loop fasteners, loops, adjustable loops, combinations of the same and like that are configured to connect to a portion of the weight transfer device, e.g., arms of the exoskeleton and/or other locations of the weight transfer device.

In one embodiment, the radiation garment is formed by attaching one or more layers together, e.g., stitching the first outer layer, the first inner layer, and the at least two first core materials together. The stitching may include stitching the second outer layer, the second inner layer, and at least two second core materials together. In various embodiments, the stitching may include stitching the third outer layer, the third inner layer, and at least one third core material together. In one embodiment, layers of the radiation garment are attached together by seaming each of the layers. Optionally and/or alternatively, shoulder seaming each of the first layers, second layers, and third layers can be done. Optionally and/or alternatively, the radiation shielding garment may include a plurality of arm guards, a short, a pant, and/or undergarments. The core materials are described herein as radiation shielding materials and include any radiation shielding material described herein, e.g., a material configured to attenuate, e.g., block radiation waves or radiation. Of course, other high-density materials that exhibit high attenuation of radiation can be utilized and these radiation materials are described herein. In one exemplary embodiment, the core material includes 0.25 mm lead equivalence.

In one embodiment, the radiation shielding garment may include an active cooling system configured to provide heat transfer to a user using the garment. The active cooling includes a heat exchanger with a heat exchanger fluid. For example, the heat exchanger can include a variety of tubes arranged as an inner material or another location. In yet another embodiment, the heat exchanger active cooling device includes forced convection provided by a fan, ventilation with a fan or other device.

In another embodiment, the radiation shielding garment may include a passive cooling system or unit. A passive cooling system may be, a cooling material configured to dissipate heat, e.g., a foil material lining coupled or otherwise integrated into the radiation shielding garment.

In another embodiment, the radiation shielding garment can include an electronic device or other type of device for monitoring radiation shielding garment usage, such as a dosimeter. A dosimeter can measure exposure to radiation. The dosimeter may be worn as a badge or a bracelet and can include phosphor crystals capable of trapping electrons freed by harmful ionizing radiation. The dosimeter can be permanently attached to the radiation shielding garment or releasably attached. When heated, the crystals release trapped electrons in the form of light—which can be measured to determine how much radiation the meter and its wearer have been exposed to. Dosimeters can be used by researchers, maintenance staff and anyone else working in a potentially radioactive environment.

In another embodiment, a usage monitoring device may be provided, for example, in the form of one or more sensors disposed throughout the radiation shielding apparatus. The usage monitoring device is configured to measure the number of times the radiation garment is used by a user. In another embodiment, usage monitoring may be facilitated, for example, by a vinyl window integrated into the radiation shielding garment, wherein the radiation shielding garment usage may be displayed.

In one embodiment, the wearable weight transfer device is configured to support the weight of a radiation shielding garment which is described herein or other radiation shielding apparatus, such as a lead apron, conventional radiation shielding apparatus, or other heavy garment. The weight transfer device is configured to transfer the weight of the garment that would normally be supported by the wearer's shoulders and/or neck, e.g., by the user's hips.

In one embodiment, components of the weight transfer device includes a first shoulder extension, a second shoulder extension, an upper back plate, a lower back plate and a waist belt. The first shoulder extension, the second shoulder extension, the upper back plate, lower back plate and attachment member can be constructed from various materials, e.g., plastic, reinforced plastic, polymers, thermoplastics, composite materials, metals, alloys, aluminum, aluminum alloy, stainless steel, combinations of the same and the like. In one embodiment, it is configured to be lightweight and very strong with reinforced plastic materials. In another embodiment, portions of the weight transfer device can be partially removed to reduce weight.

The radiation shielding garment, in one embodiment, includes a vest, a skirt and a thyroid shield. Optionally, the vest is configured with attachment mechanisms to allow it to be attached to the weight transfer apparatus at one or more attachment points. The attachment mechanism can be magnets, snaps, hook and loop fasteners, fabric loops, mesh, combinations of the same the like.

In one embodiment, the weight transfer apparatus is directed towards a fixed-angle, wearable device that supports the weight of a heavy garment, such as a lead apron or other heavy garment as described in U.S. Pat. No. 10,729,195, which is hereby incorporated by reference for such teachings. The weight transfer device transfers the weight of the radiation shielding garment that would be supported by the wearer's shoulders and/or neck to the wearer's hips without a weight transfer apparatus. The weight transfer apparatus at least partially transfers weight off the shoulders of a user, so that there is less pressure on the cervical spine of a user. Many radiologic procedures require the user to bend forward for significant periods of time. At the same time, the radiation shielding apparatus aids in correcting the user's posture and/or to support a user's lower back, thereby alleviating weight and pressure that over time can cause lower back injuries. The apparatus also creates space between the wearer's body, particularly their shoulders and the backs, and the radiopaque garment being worn and, as a result, helps to provide ventilation within the garment while in use and aids in cooling the user.

In another embodiment, the weight transfer support system generally includes three components: a hip or waist belt, a back support, and one or more shoulder extensions. The shoulder extensions include one or more of rigid bars, slabs or blocks of material that attach to the back support and extend away from the back support and above the wearer's shoulders when the device is worn. The shoulder members are adjustable in shape to mimic a user's dimensions.

In one embodiment, the weight transfer apparatus includes protrusions or pins located on a surface of the shoulder extensions so that the pins protrude away from the shoulder extensions and the wearer. The protrusions can also be used to attach other devices, such as a splash shield, face shield, and/or radiation garment shield apparatus.

In one embodiment, the weight transfer apparatus can include non-slip material or non-slip coatings on one or more portions of the shoulder extensions, including but not limited to a location that is most likely to come into contact with the radiation shielding garment.

In one embodiment, the weight transfer apparatus includes shoulder extensions that are flattened, curved metallic bars that attach at one end to an upper portion of the frame and extend up and over the user's shoulders when the device is worn. The shoulder extensions are attached to a back support that generally includes an upper back plate, a lower back plate, and an attachment bar. In a preferred embodiment, the shoulder extensions are attached to the upper back plate at an angle to the center of the upper back plate such that they extend outward away from the user's head as they extend over the user's shoulders. Optionally and/or alternatively, the device can be constructed so that the shoulder extensions are joined permanently with the back plate to form one unitary structure. In addition, several mechanisms of attachment can be used to secure the shoulder extensions to the upper back plate and via holes or threaded bores passing through the upper back plate. The shoulder extensions could be used to accommodate a variety of fasteners to accomplish this purpose. Moreover, when the shoulder extensions are releasably attached to the back support, the plurality of holes on both the shoulder extensions and the upper back plate allow the user to reposition the shoulder extensions in relation to the rest of the device by using different holes to attach the shoulder extensions to the upper back plate.

In another embodiment, the weight transfer apparatus can include padding on the ventral side of the shoulder extensions, e.g., the side that faces the user when worn. In some embodiments, the padding features holes complementary to the holes in the shoulder support and the upper back plate so that the same fastener can be used to hold all three parts together at the same time. Other fasteners such as glue or double-sided tape to attach the padding to the shoulder extensions can also be used.

In another embodiment, the upper and lower back plates can each be separately attached to an upper and a lower attachment bar, respectively. In one embodiment, a hinge joins the upper and lower back plates. The upper and lower attachment bars are joined together via a hinge. In another embodiment, the apparatus can use a single rigid bar, e.g., the attachment bar, attached to the upper back plate at one end and the lower back plate at the opposing end. In addition, the lower back plate can be shaped to receive the lower end of a single attachment bar, e.g., the lower back plate is shaped to form a bracket that at least partially encircles the portion of the attachment bar inserted therein. This structure is configured to hold the attachment bar in place relative to the lower back plate and serves to create some space between the user and the face of the attachment bar adjacent to their body. In a preferred embodiment, the lower end of the attachment bar features a number of holes or threaded bores extending all the way through from one side of the attachment bar to the opposing side sized, positioned, and shaped to align with one or more complementary holes or threaded bores located in the lower back support to allow the two parts to be fastened together. These embodiments feature a fastener, preferably a retractable plunger, i.e. a spring-loaded plunger, configured to be inserted into the holes or bores in the attachment bar and the lower back plate thereby attaching the lower portion of the device featuring the lower back plate and the hip belt(s) to the upper portion that features the shoulder extensions.

In one embodiment, the weight transfer device includes a lower back plate and one or more belts designed to encircle the waist and hips of the person using the device. The mechanism of attaching the hip belt to the lower back plate is holes or threaded bores in the lower back plate that align with holes or bores in the hip belt or combinations of the same and the like. The hip belt is a padded belt that is meant to be worn around the wearer's waist area and has a buckle or clasp allowing the wearer to attach the two free ends of the hip belt.

In another embodiment, the weight transfer device can include a second belt—a stretch belt—configured to allow the user to attach the device to their hips, adjust the position of the device, and then attach the hip belt. The stretch belt is located inside the hip belt and is attached to an inner surface of the hip belt and extends around the person wearing the device so that it is between the hip belt and the person wearing the device.

Optionally and/or alternatively, the stretch belt can be made with a lumbar pad located on the portion of the stretch belt that is in contact with the lower back plate. The hip belt itself is directly attached to the lower back plate. The stretch belt can have a built in lumbar pad attached to the hip belt by hook and loop fasteners; although, other attachment mechanism could be used as well. The stretch belt can be a separate structure from the hip belt, or the two can be integrated into a single structure.

In one embodiment, the user wearing the device first adjusts the height of the device by removing the spring-loaded plunger from the complementary holes or bores in the alignment bar and lower back plate, adjusts the upper back plate to the desired height, then reinserts the plunger into the holes or bores. Similarly, in some embodiments, the position or height of the shoulder extensions can be adjusted by removing the fasteners holding them to the upper back plate, repositioning them relative to the holes or bores on the upper back plate and then reattaching the two pieces. The user then puts on the device by putting it on their back and securing the stretch belt around their waist. The wearer can then adjust the positioning of the device with respect to their body and when the device is properly positioned they secure the hip belt around their waist.

In one embodiment, once the user is wearing the weight transfer device, the radiation shielding device, e.g., lead apron or other garment is attached to the device so that the garment is worn over the weight transfer device. The radiation shielding garment may also be integrated into the weight transfer apparatus as described herein. The shoulder extensions can fit into sleeves featured by the garment; the typical lead apron used by medical professionals often has sleeves already integrated into it. In addition, nonslip pads featured on the outer facing portions of the shoulder extensions help to hold the garment in place by creating friction between the garment and the nonslip pads.

In one embodiment, after the hip belt is secured around the waist and the garment is attached or draped over the device, the weight of the radiation shielding garment and the device does not rest on the wearer's shoulders or spine, but is transferred to their hips. The lower and upper back plates are shaped so that the garment shielding device, when the wearer stands upright, has at least a portion that does not contact the user's back, neck and/or shoulders. The positioning of the padding can also assist in holding the radiation shielding garment in place on the user and creating space between the garment and the user. The space between the user and the radiation shielding garment allows air to enter through the sides or top of the apron or garment, to circulate air and keep the wearer cooler than they would be if they wore the garment against their person. The weight transfer device thereby allows the wearer to perform lengthy medical procedures, even those that require bending over the patient for prolonged periods of time, without having to support the weight of the required radiation shielding garments with their shoulders or backs.

In one embodiment, the weight transfer device includes shoulder extensions having a width in range from about 0.25 inch to about 2 inches or greater, a thickness in range from about 0.125 inch to about 0.5 or greater, and length in range from about 6 inches to about 16 inches or greater. In a preferred embodiment, the shoulder extensions are about 1.5 inches wide and made of 6061 aluminum bars that are flattened to approximately 3/16 of an inch thick. In another preferred embodiment, stainless steel #10-24 binding barrels and #10-24 stainless steel machine screws are inserted through complementary holes in the shoulder extensions and the upper back plate to connect each shoulder support to the upper back plate. In another preferred embodiment, an elastomer compound, Regupol #7210, can be used to form the nonslip pads on the shoulder extensions and/or upper or lower back plates. In yet another preferred embodiment, Poron 4701-40 soft rubber approximately 0.50 inches thick is used with a piece of approximately ABS plastic for spacing and attachment to form the pads on the shoulder extensions and/or upper or lower back plates. In yet another preferred embodiment, the upper and lower back plates are vacuum formed pieces of Boltaron 4335 plastic that are approximately inches thick. In yet another preferred embodiment, the attachment bar is attached to the upper and lower back plates with #10-24 stainless steel machine screws and nylon lock nuts. The attachment bar itself is approximately 1.50 inches wide and approximately 0.188 inches thick and is made of 6061 aluminum rectangular bar stock. In such embodiments the length of the attachment bar can be varied to increase or decrease the size or height of the overall device as desired. These same embodiments use steel binding barrels having a ¼ inch—20 thread and are ⅜ inches long to attach the hip belt to the lower back plate. In yet another preferred embodiment, the hip belt includes two grommets with an inner diameter of approximately 0.3125 inches. Approximately in this context means within 0.25 inches of the stated measurement. The spring-loaded plunger can take a number of forms, including a twist-to-lock pull-ring retractable spring plunger. The weight transfer device can have other dimensions and use other materials as described herein.

In one embodiment, the weight transfer apparatus allows the user to use/wear radiation shielding garments, e.g., heavy radiation shielding garments, without obstructing the function of those radiation garments or the user's movement. For example, the weight transfer apparatus allows a user to wear various types of heavy clothing or equipment without interfering with the normal movements of the user. The apparatus also provides lumbar support and improves body ventilation for those wearing heavy shielding garments. By transferring weight away from the shoulders, the device reduces the incidence of cervical orthopedic injury sustained by the user. By providing lumbar support to the user, the weight support also results in improved posture and reduces the incidence of lower back strain and injury. Moreover, because the weight support creates some separation between the clothing and the user, the weight support serves to provide additional ventilation that is not available when the user wears the same clothing or equipment directly against their body. The device is lightweight, comfortable, durable, easy to clean, and easy to put on or remove. In addition, since the device is not integrated into a garment, the device can be used with more than one garment including a “half-garment” that is meant to be worn only over the front of the wearer. In such embodiments, the back support and other components of the device can be made of or infused with radiation shielding materials, e.g., radio-opaque materials to help increase the shielding provided by the garment.

In one embodiment, a face shield is configured to be attached to the apparatus. The face shield can be an accessory that can be used with the weight transfer support device described herein. The various embodiments of the face shield can be used with the disclosed weight transfer support device or with other frames. Generally, the face shield is made with radiation shielding materials, i.e., a radio-opaque material that is visually transparent and allows the wearer to see through the shield but blocks or substantially blocks the radiation used in medical procedures, e.g., x-ray radiation.

In one embodiment, the face shield is constructed from a lead acrylic material configured to be visually transparent while blocking radiation. The face shield can have a thickness in a range from about to about and is configured to at least partially or fully cover a user's face. In a preferred embodiment, the face shield has a thickness ranging from about 3/16 inch to about 5/16 inch or greater. The face shield can include tempered glass with polished edges. Other materials radiation shielding materials and/or coatings can also be utilized, e.g., lead glass.

In one embodiment, the face shield is attached to a yoke mount that also connects to the weight transfer support system described herein and/or the weight transfer device described in U.S. Pat. No. 10,729,195, which is hereby incorporated by reference. The yoke mount is a structure, such as a metal bar, to which the face shield and a back attachment are connected.

In a preferred embodiment, the face shield is attached to a shield mount, which is a structure featuring an attachment mechanism that attaches the face shield to the yoke mount. In some preferred embodiments, the shield mount is a block with holes to accept fasteners that is also fixed to the face shield using conventional fasteners such as screws. It is not necessary that the shield mount be releasably attached to the face shield. The shield mount is releasably connected to a portion of the yoke mount by a separate fastener such as a thumb screw. That is, the shield mount features a channel or bore or similar structure through or with which a portion of the yoke mount passes or engages and is held in place using pressure applied via a fastener such as a thumb screw. This arrangement allows the user to adjust the position of the face shield relative to the yoke mount by manipulating the thumb screw or other fastener holding the shield mount to the yoke mount to loosen the connection between the yoke mount and the shield mount, repositioning the shield mount relative to the yoke mount and then tightening the fastener.

In one embodiment, the yoke mount can include one bar or a similar durable structure that is configured to extend around the user's head on either side. These embodiments of the yoke mount include a rigid bar with a rounded exterior that has two vertical portions at opposite ends connected by a horizontally oriented portion that extends over each shoulder and behind the user's head and neck. The vertical portions of the yoke mount extend upward, away from the tops of the user's shoulders and/or the shoulder extensions, and are adjacent to the side of the user's head. The face shield mount is configured to releasably connect the face shield to this vertical portion of the yoke mount, thereby allowing the vertical position of the face shield to be easily adjusted up and down. The yoke mount could be composed of separate pieces, one for each side of the user, or one single piece that extends around both sides of the user's head or other configurations.

In one embodiment, the face shield extends around the face of the wearer and connects to and is held in place on the yoke mount on either side by the shield mounts units that are releasably connected to the yoke mounts on either side of the face shield. Because the yoke mount is able to rotate, the face shield is able to move with the user when they move their head or lean forward. In this embodiment, the face shield rests at least partially on the shoulder extensions of the shoulder weight transfer device allowing the shoulder extensions to support the full weight of the face shield. In another embodiment, the face shield can be mounted eliminating the yolk mount as described herein. In this embodiment, the face shield is configured to attach to at least one of the shoulder extensions of the shoulder transfer weight support system thereby allowing the shoulder extensions to support the full weight of the face shield. The yoke is replaced with a vertical mount bar and a horizontal slide bar which can be separate structures attached to each other or they can be made as one single structure. The vertical mount bar attaches to the face shield in the same manner as the vertical portion of the yolk in the embodiments described above. That is, the shield mount features a channel or bore or similar structure through or with which a portion of the vertical mount bar passes or engages and is held in place using pressure applied via a fastener such as a thumb screw. The horizontal slide bar is attached to the vertical mount bar. The angle and point of attachment between these two structures can be varied as desired. In these embodiments, the horizontal slide bar is attached to the shoulder extensions of the shoulder transfer weight support via a shoulder extension mount. Preferably, the shoulder extension mount is releasably connected to the horizontal slide bar so that the position of the face shield can be adjusted in a horizontal plane in addition to a vertical plane. in a preferred embodiment, a thumb screw releasably attaches the shoulder extension mount to the horizontal slide bar. While the shoulder extension mount can also be releasably attached to the shoulder extension, the inventors anticipate that the most practical version of this device will involve using friction to keep the shoulder extension mount in place as it is shaped to provide a groove into which the shoulder extension, together with its padding, and the radio-opaque garment to be worn on the shoulder extensions is inserted. Other embodiments of the shoulder extension mount include means to adjust the width of the groove featured by the shoulder extension mount to provide for a tighter fit. The inventors specifically anticipate this device will be used with the shoulder transfer weight support system described above and/or in the '235 Application; however, all of the embodiments above can be mounted to other devices if desired.

Still other embodiments of the inventive face shield include a face shield that does not extend completely around the user's face. The above embodiments anticipate that the user will need to protect themselves from radiation that may emanate towards either side of their head or body. Alternate embodiments of the face shields have an open front. For example, in one embodiment, the face shield extends along the side of the user's head and terminates in a curved shape that bends toward the user's face, but stops short of extending all of the way in front of it and/or covering the user's mouth. Another example would include an entirely flat face shield that extends only laterally along the side of the user's head, but does not include a curved portion that extends towards or in front of the user's face.

The terms such as horizontal, upward, vertical, above, below, beneath, and the like are used solely for the purpose of clarity in illustrating the invention, and should not be taken as words of limitation. The drawings are for the purpose of illustrating the invention and are not intended to be to scale. Generally, references to upward directions, including “above” and “over” indicate a direction towards the wearer's head when the device is being worn. Directions indicating a downward direction including “beneath” and “under” indicate a direction toward the wearer's feet when the device is being worn. “Ventral” refers to a direction toward the wearer's abdomen while the device is being worn. “Dorsal” refers to a direction towards the back support of the device.

Reference will now be made in detail to various embodiments of the invention, examples of which are illustrated in the accompanying drawings.

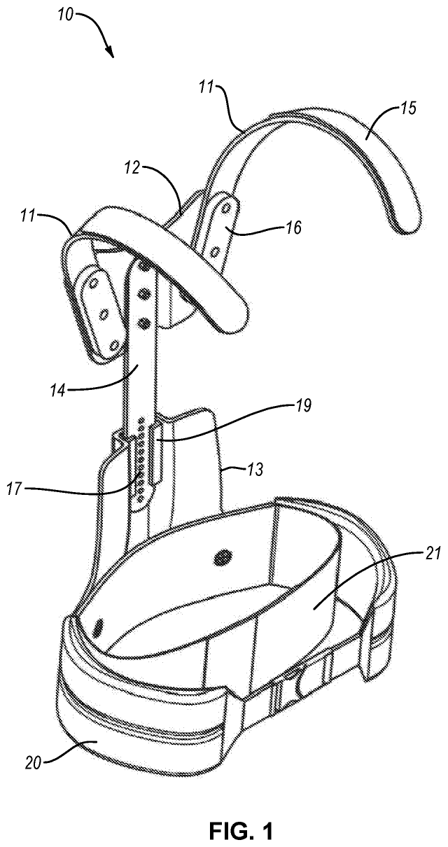

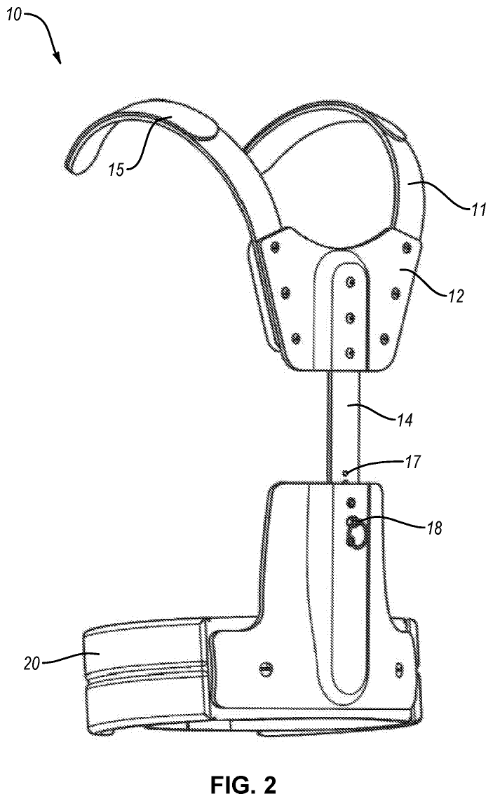

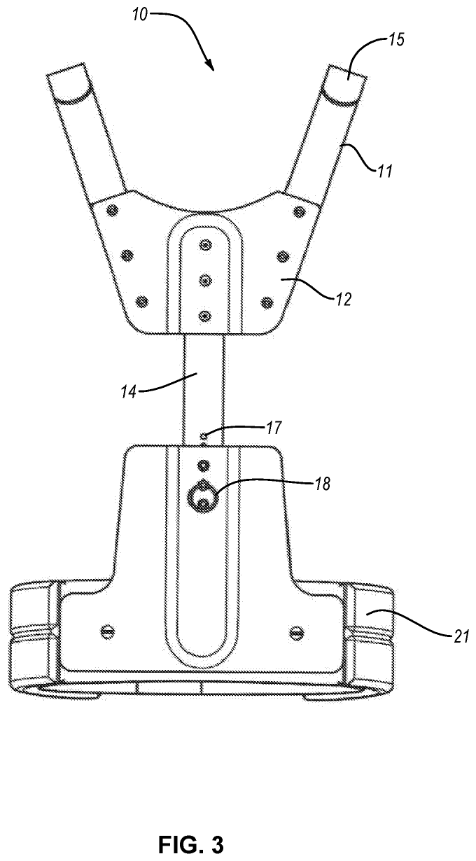

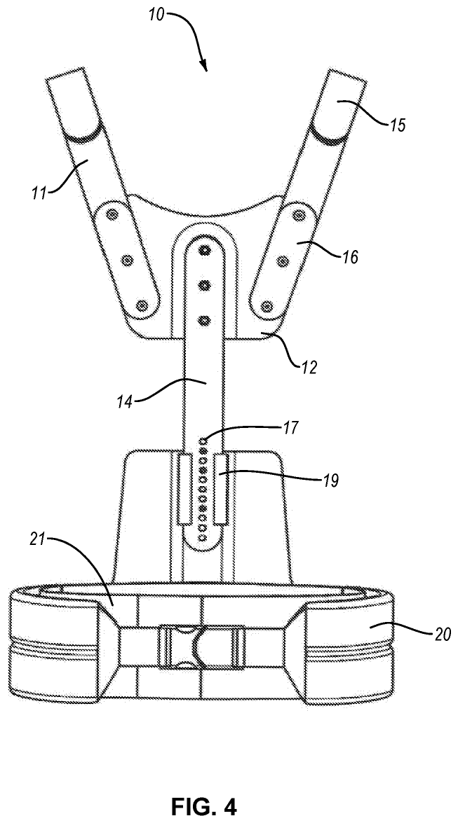

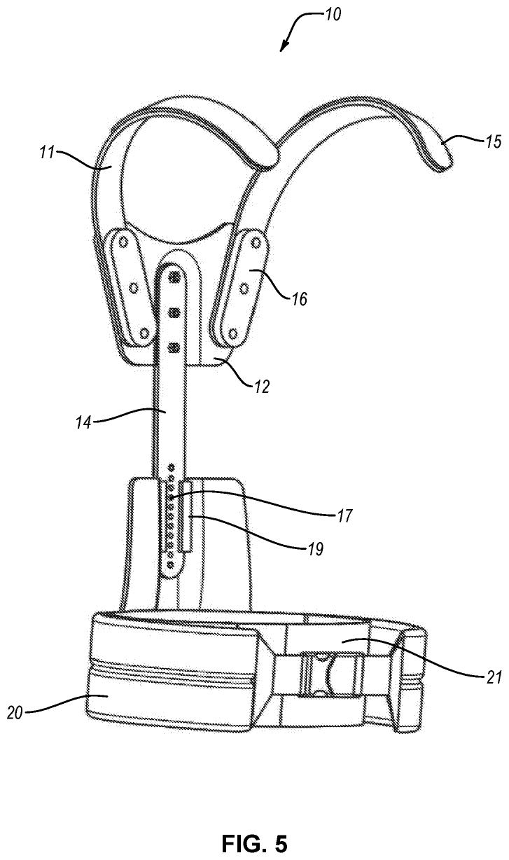

FIG. 1 is a left side perspective view of a weight transfer support device according to an embodiment of the invention. FIG. 2 is a rear right side perspective view thereof. FIG. 3 is a back perspective view thereof. FIG. 4 is a front perspective view thereof. FIG. 5 is a left side perspective view thereof.

Referring to FIGS. 1-5, the weight transfer support device is generally depicted with reference to number 10. The weight transfer support device 10 is shown without a face shield attached or a radiation apparatus attached or utilized. The weight transfer support device 10 includes two shoulder extensions 11 extending upward and away from the upper back plate 12 to which they are attached. The shoulder extensions 11 can be attached to the front side of the upper back plate 12 or to the back side of the upper back plate 12. The shoulder extensions 11 have an outer surface that faces upward and away from the user's shoulders when the device 10 is worn. The outer surface of the shoulder extensions can feature nonslip pads 15 to help hold a garment or radiation shielding apparatus (not shown) as described herein in place. The shoulder extensions 11 can be constructed from metal, plastic, polymer, carbon fiber, alloy, aluminum, combinations of the same and the like. In this embodiment, the shoulder extensions 11 are constructed from 6061/T6 aluminum.

Optionally and/or alternatively, padding 16 can be attached to the upper back plate 12 and/or the shoulder extensions 11. The padding 16 can be positioned so it will be in contact with the wearer when the device 10 is worn. The upper back plate 12, an attachment bar 14 and the padding 16 all feature complementary holes that allow for the insertion of fastening devices to connect the various parts as described above.

The attachment bar 14 is attached to the upper back plate 12 at a first end and a lower back plate 13 at a second end. The lower end of the attachment bar 14 features a plurality of holes 17 that are complementary in size and shape and align with one or more holes that pass through a portion of the lower back plate 13. The position of the upper back plate 12 and shoulder extensions 11 can be adjusted as follows. The attachment bar 14 can be held in place using a fastener 18 inserted through the holes 17 in the lower back plate 13 and the lower end of the attachment bar 14. With the fastener 18 removed, the attachment bar 14 can be positioned such that different holes in the attachment bar 14 are aligned with holes in the lower back plate 13 thereby adjusting the overall height of the device 10. In a preferred embodiment, fastener 18 is a retractable plunger, e.g., a spring-loaded plunger such as a twist-to-lock pull-ring retractable spring plunger, to allow for rapid adjustment.

The lower back plate 13 has a section that is configured to accept or accommodate the lower end of the attachment bar 14. In the embodiment shown in these figures, the upper edge of the lower back plate 13 is shaped like an open-faced channel or bracket 19 into which the lower end of the attachment bar 14 inserts. Note, in the pictured embodiment, the portion of the attachment bar 14 that features the holes 17 is recessed, i.e. the attachment bar is not flush with the inner surface of the lower back plate 13. This allows for the insertion of the plunger 18 without the end of the plunger 18 sticking the person wearing the device 10 in the back. It also provides space between the user and the device so that air can circulate next to the wearer's body.

The lower back plate 13 also features one or more belts. This embodiment has two belts: a hip belt 20 and a stretch belt 21. The hip belt 20 is directly attached to the lower back plate 13 while the stretch belt is connected to the hip belt 20 either using the same fasteners inserted through the lower back plate 13 and the two belts 20, 21, or using separate fasteners attached to the hip belt 20. In one embodiment, hook and loop fastener (not shown) can be utilized to attach the stretch belt 21 to the hip belt 20. Of course, other attachment mechanisms can also be used, e.g., buckles, snaps, magnets and combinations of the same.

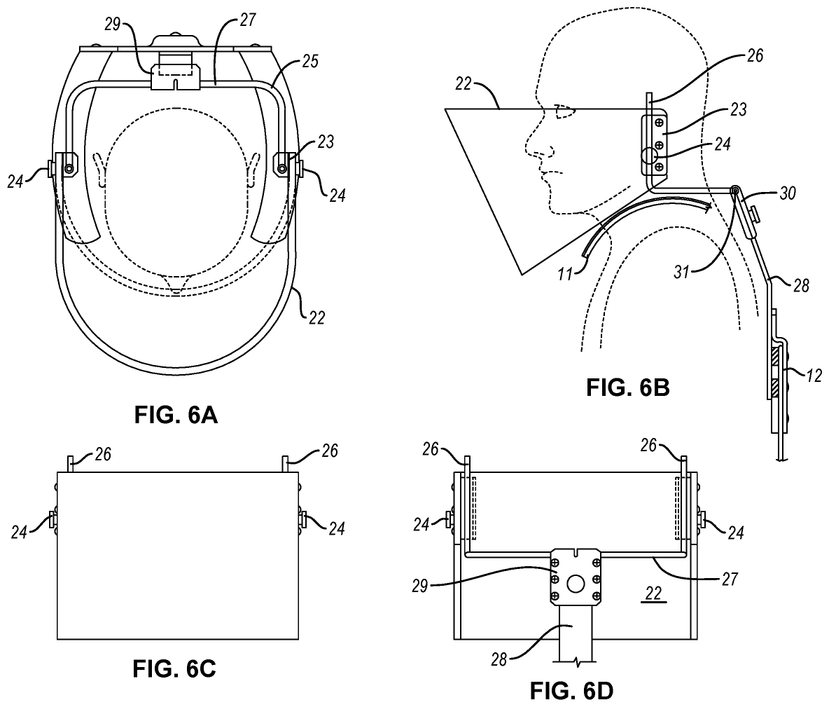

FIG. 6A is a top perspective view of a weight transfer support system featuring a face shield according to another embodiment of the invention. FIG. 6B is a right side perspective view thereof. FIG. 6C is a front perspective view thereof. FIG. 6D is a rear perspective view thereof.

Referring now to FIGS. 6A through 6D, a face shield 22 is shown. In this embodiment, the face shield 22 extends from one side of the user's head to the other, providing coverage for the user's entire facial area. The face shield 22 includes a shield mount 23 that is fixed to the face shield 22. The shield mount 23 in this example is a block featuring bores or holes into which fasteners can be inserted to attach the shield mount 23 to the face shield 22. In a preferred embodiment, the shield mount is constructed of plastic, e.g., Delrin plastic (Acetal). In this embodiment, the shield mount 23 includes thumb screws 24 or other mechanisms configured to releasably attach the shield mount 23 to a vertical section 26 of a yoke mount 25.

In this embodiment, the yoke mount 25 includes a cylindrical tube or bar featuring a vertically oriented section 26 at each of the two ends of the yoke mount 25 and a horizontally oriented section 27 connecting the two vertical sections 26. The yoke mount 25 is positioned such that the horizontal section 27 wraps around the back of the user's head as it extends from one vertical section 26 to another.

The user can adjust the position of the face shield 22 relative to their face or the vertical section 26 of the yoke 25 by loosening the thumb screw 24, moving the face shield 22 up or down on the vertical section 26 and then manipulating the thumb screw 24 to tighten the connection between the yoke mount 25 and the face shield 22.

The face shield 22 attaches to the weight transfer device 10 described above through the horizontally oriented section 27 of the yoke mount 25 attached to a back attachment bar 28. The back attachment bar 28 is a flattened structure that is attached to and extends upward and away from the upper back plate 12 of the weight transfer device 10 described above. It terminates in a machined fitting 29 that accommodates and holds a portion of the horizontal section 27 of the yoke mount 25. In the pictured embodiment, the machined fitting 29 consists of two plates 30 joined together by a fastener that form a small channel or groove 31 into which the yoke mount 25 fits. The plates 30 are tightened together to encircle a portion of the horizontal section 27 of the yoke mount 25. This configuration also allows the yoke mount 25 to rotate around the longitudinal axis of the horizontal section 27 of the yoke mount 25, specifically the longitudinal axis of the portion of the yoke mount 25 that is attached to the back attachment bar 28. This rotational movement allows the face shield 22 to tilt when the user tilts their head forward. It also allows the user to move the face shield 22 out of their way by lifting the face shield 22 up and over their head, allowing the face shield 22 to rest against the back of the weight transfer device 10 when not needed.

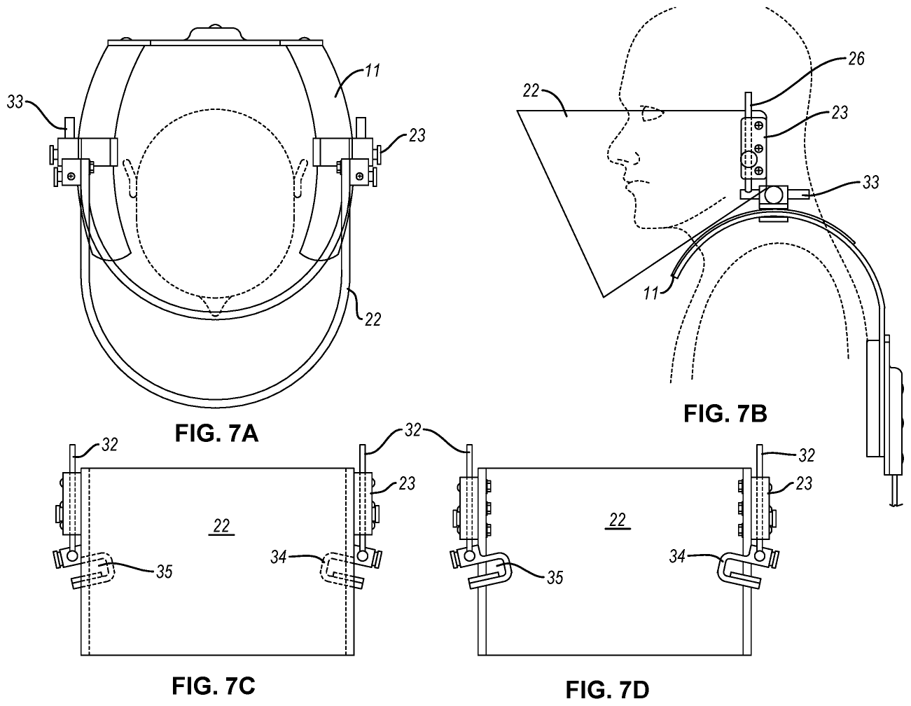

FIG. 7A is a top perspective view of an embodiment of a weight transfer support system including a face shield according to another embodiment of the invention. FIG. 7B is a right side perspective view thereof; FIG. 7C is a front perspective view thereof. FIG. 7D is a rear perspective view thereof. FIGS. 7A through 7D illustrate another embodiment of the face shield.

Referring now to FIGS. 7A-7D, in this embodiment, the yoke mount is replaced with a vertical mount bar 32 attached to a horizontal slide bar 33 with a mount configured to attach to the shoulder extensions 11 of the weight transfer device 10. The face shield 22 is attached to the vertical mount bar 32 in much the same way as the face shield in the embodiments above was attached to the vertical section 26 of the yoke mount 25, e.g., the shield mount 23 in this embodiment works the same way as it does for the above embodiment. The mount or shoulder extension mount 34 is releasably attached to the horizontal slide 33 using a thumbscrew 24 or other attachment means. The position of the face shield 22 relative to the user can be adjusted up and down the vertical mount bar 32. The thumb screw 24 on the shoulder extension mount 34 can be loosened to allow for repositioning of the horizontal slide bar 33 relative to the shoulder extension mount 34 thereby adjusting the position of the face shield 22. In this particular embodiment, there are vertical mounts 32, horizontal slide bars 33 and shoulder extension mounts 34 on either side of the user/weight transfer device to allow for the positioning of face shield 22 on both sides of the user.

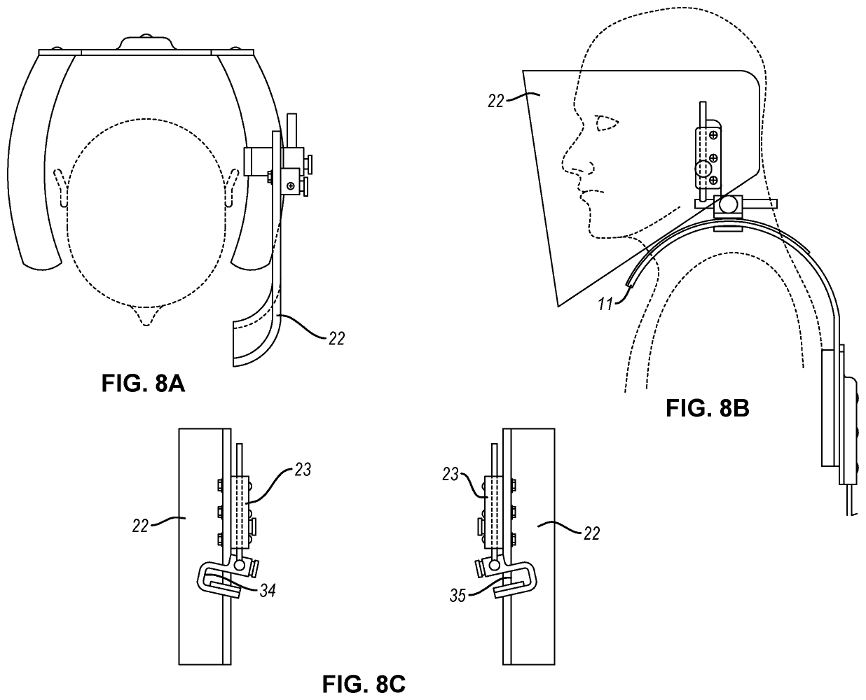

FIG. 8A is a top perspective view of an embodiment of a weight transfer support system featuring a face shield according to another embodiment of the invention. FIG. 8B is a right side perspective view thereof. FIG. 8C is a front perspective view thereof.

Referring to FIG. 8A-8C, another embodiment, e.g., an alternate version of the face shield 22 as used with the above-described embodiments is illustrated. In this embodiment, the face shield can be used with either the yoke mount 25 or the shoulder extension mount 34. Because it is common for medical professionals to have difficulty communicating with other people in the room while they are wearing a face shield that covers a significant portion of their mouth and the source of radiation in an operating room is often located to the side of the surgeon, the inventors have devices other face shields that do not cover their entire mouth. For example, FIG. 8A shows a partial lead acrylic shield 22 that is open in the front and bends toward the other side of the user as it extends past the user's face.

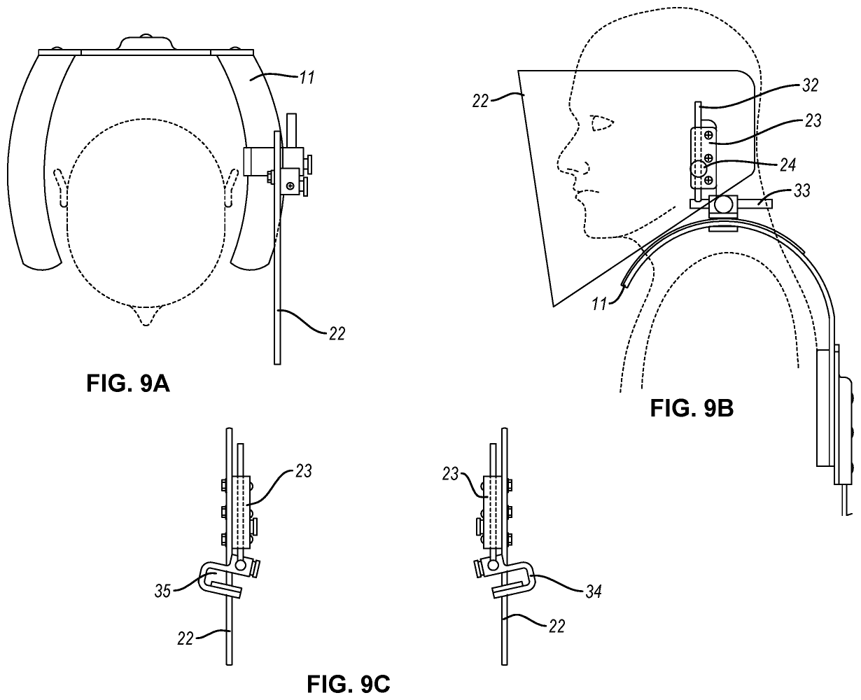

FIG. 9A is a top perspective view of an embodiment of the weight transfer support system with a face shield according to another embodiment of the invention. FIG. 9B is a right side perspective view thereof. FIG. 9C is a front perspective view thereof.

Referring to FIGS. 9A-9C, in these embodiments the face shield 22 is a flat piece of material that extends along the side of the user's head only. These iterations of the face shield 22 allow the surgeon to more easily communicate with others in the room while staying shielded from a radiation source that is not directly in front of them.

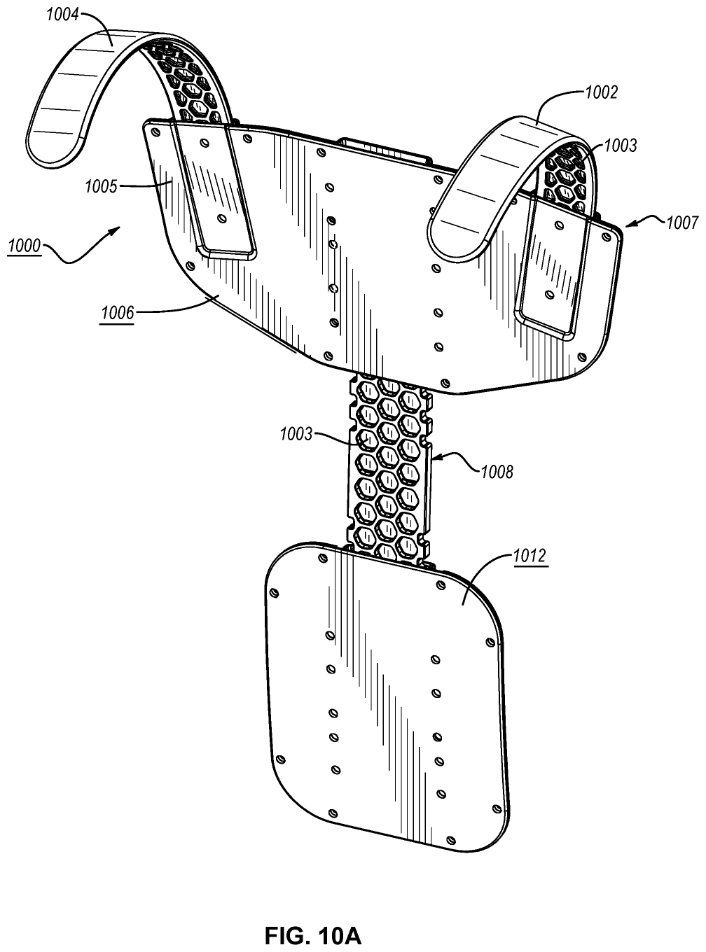

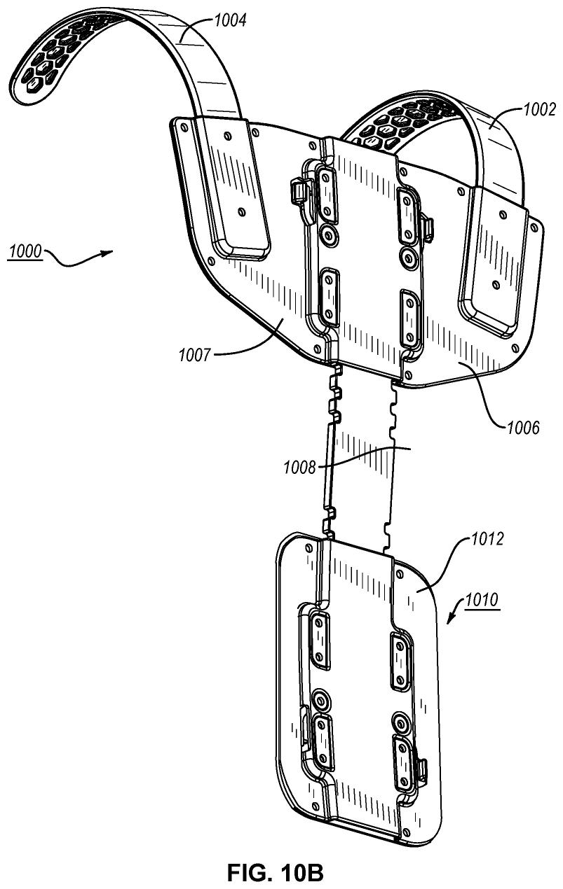

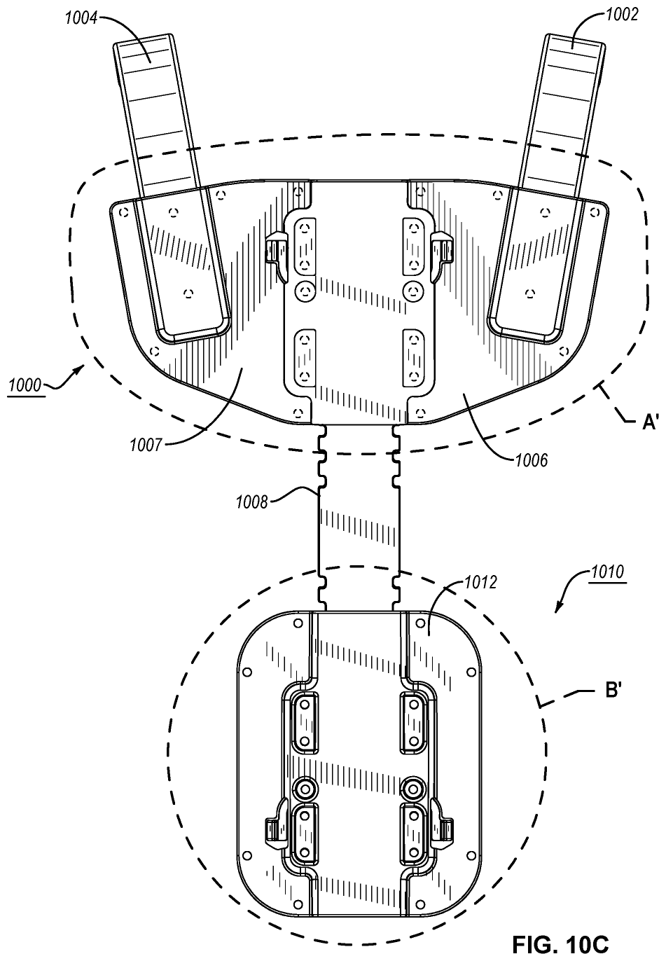

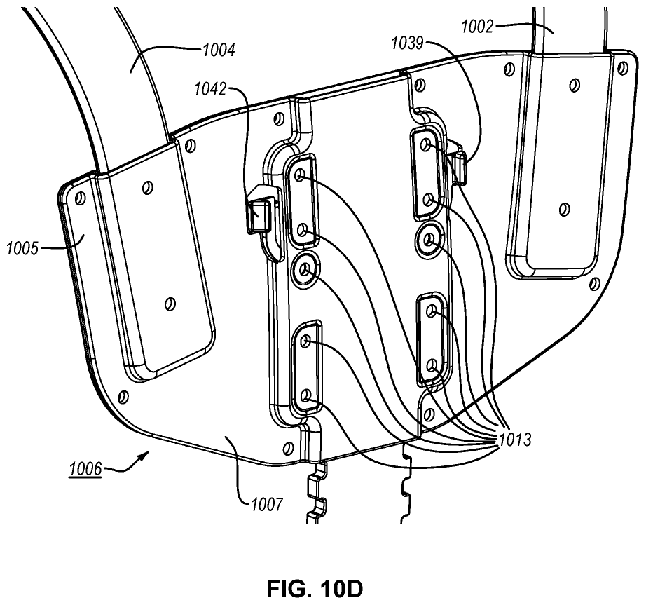

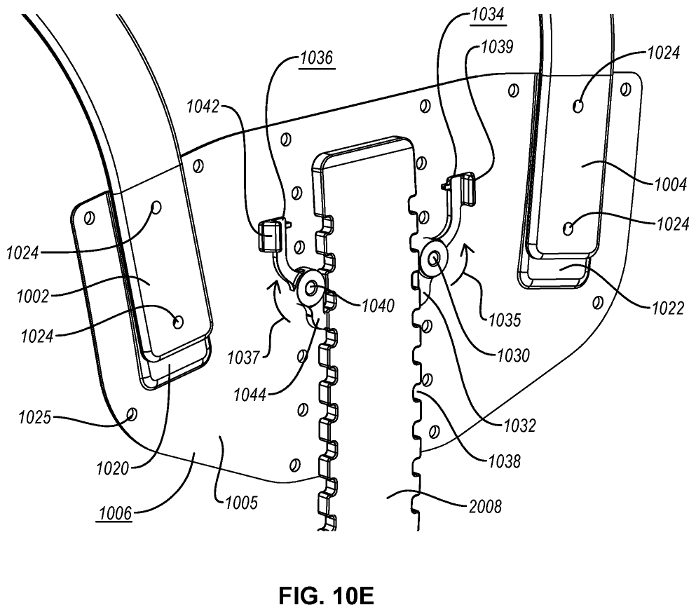

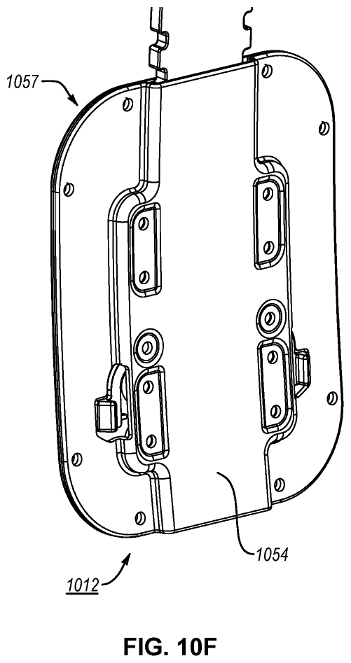

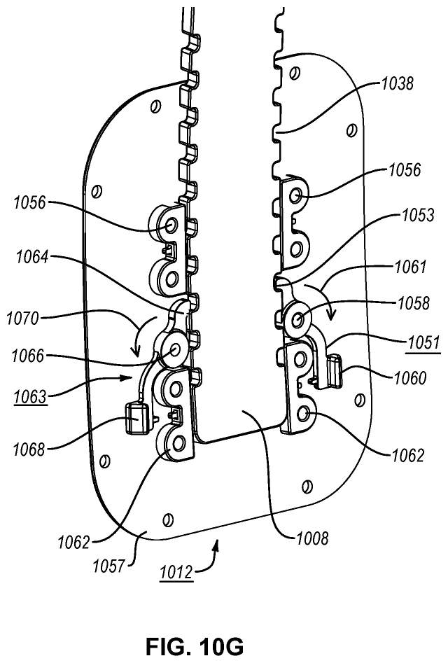

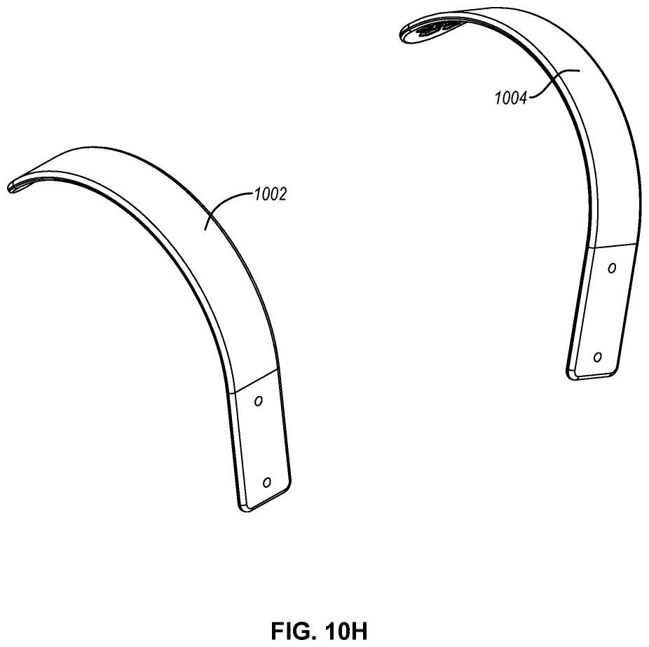

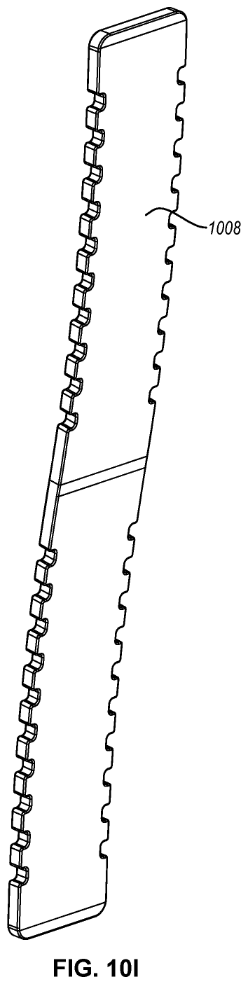

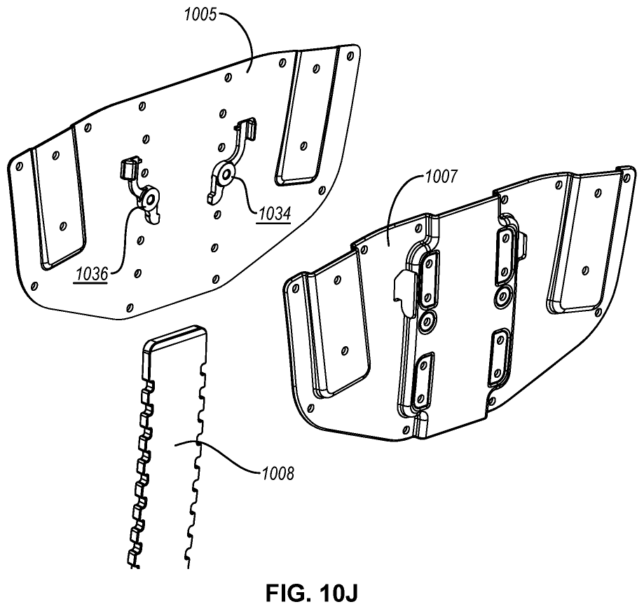

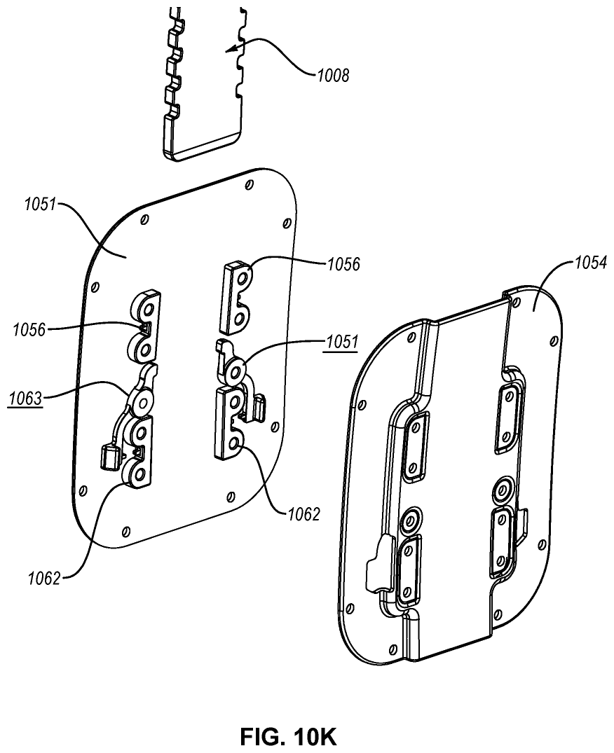

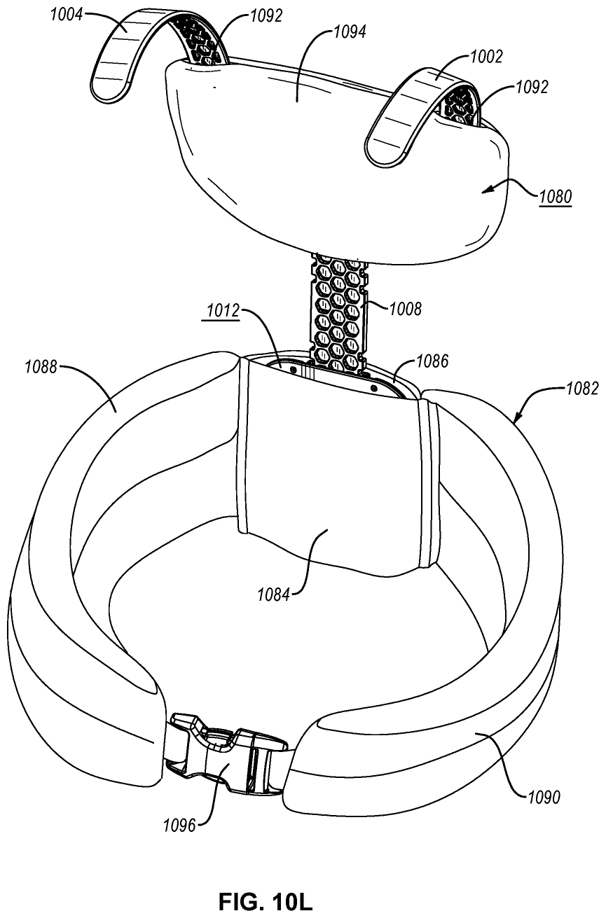

FIG. 10A is a right side front perspective view of a shoulder transfer weight support system according to another embodiment of the invention. FIG. 10B is a right side back perspective view thereof. FIG. 10C is a back perspective view thereof.

Referring to FIGS. 10A-10C, the weight transfer support device is generally depicted with reference to number 1000. The weight transfer support device 1000 includes a first shoulder extension 1002 and a spaced apart second shoulder extension 1004, each extending upward and away from an upper backplate 1006. The upper backplate 1006 is adjustably connected to a lower backplate 1012 with an attachment member 1008. In one embodiment, the lower backplate 1012 includes a first waist member (not shown) extending outwardly in a curved configuration. The lower backplate 1012 is configured to couple to the waist of a user as described herein. In addition, the upper backplate 1006 can include padding as described herein.

The shoulder extensions 1002, 1004 can be constructed from metal, plastic, polymer, fiberglass, carbon fiber, alloy, aluminum, combinations of the same and the like. In this embodiment, the shoulder extensions 1002, 1004 are constructed from 6061/T6 aluminum.

The upper backplate 1006 and lower backplate 1012 can be contrasted from a plastic, thermoformed plastic, polymer, metal, alloy, composite, e.g., fiberglass, combinations of the same and the like. In this embodiment, the upper backplate 1006 and lower backplate 1012 are constructed from Lower-Kydex a type of thermoplastic material.

The first shoulder extension 1002 and second shoulder 1004 can be attached to a front side of the upper backplate 1005 (not shown), a back side of the upper backplate 1007 (not shown) and/or between the front side of the upper backplate 1005 and the back side of the upper back plate 1007 as shown in this embodiment.

In order to decrease the weight of the shoulder extension, one or more surfaces of the first shoulder extension 1002 and the second shoulder extension 1004 can have material removed to decrease weight while substantially maintaining strength in a predetermined geometric pattern, e.g., circular pattern, honeycomb pattern and any other geometric pattern. The material can be removed by forming holes, partial holes, partially removing material, partially removing a thickness of the material, combinations of the same and the like. In this embodiment, the material is partially removed in a honeycomb pattern 1003 on only one surface of each of the shoulder extensions 1002 and 1004 and attachment member 1008. The material can be removed as a bore through the entire thickness of the material or partial bore through only a portion of the thickness. Each of the shoulder extensions 1002, 1004, can be constructed from any material as described herein. In this embodiment, each of the shoulder extensions 1002, 1004, is formed using an aluminum alloy. Each of the shoulder extensions 1002, 1004 has a curve configured to go over a shoulder of a user.

Each of the shoulder extensions 1002, 1004 has an outer surface that faces upward and away from the user's shoulders when the device 1000 is worn. The outer surface of the shoulder extensions can feature nonslip pads or a frictional coating (not shown) to aid with preventing or substantially preventing a radiation shielding garment described herein from moving when the radiation shielding garment is used with the weight transfer device 1000.

Optionally and/or alternatively, a padding material or padding coating (not shown) can be attached to the front side upper back plate 1005, front side of the lower back plate 1012 and/or inner surface of the shoulder extensions 1002, 1004. The pads and/or coating can be sized and positioned so they will contact the wearer when the device 1000 is worn. The front side upper back plate 1005 and the back side upper backside 1007 feature complementary holes 1025 that allow for the insertion of fastening devices to connect the plates together and/or connect various parts, e.g., radiation shielding garment to the apparatus 1000.

FIG. 10D is a magnified view of portion A′ of FIG. 10C. FIG. 10E is a partially disassembled view of FIG. 10D. FIG. 10F is a magnified view of portion B′ of FIG. 10C. FIG. 10G is a partially disassembled view of FIG. 10F. FIG. 10H is a perspective view of the shoulder extensions. FIG. 10I is a perspective view of an attachment member. FIG. 10J is a partially disassembled perspective view of an attachment member and the upper back plate. FIG. 10K is a partially disassembled perspective view of an attachment member and lower back plate thereof.

Referring to FIG. 10D-10K, the back side upper backplate 1007 has been removed. An attachment member 1008 includes a plurality of side notches 1038 spaced at predetermined intervals to aid with adjusting the distance of the upper backplate 1006 from the lower backplate 1012. Mounted on an interior surface of the front side upper backplate 1005 is a first adjustable attachment mechanism 1034 and a second adjustable attachment mechanism 1036. The first adjustable attachment mechanism 1034 includes a biasing member 1030, e.g., a spring or other biasing means, attached to front side of the upper back plate 1005, a first extension extending from the biasing member 1030 with a tab 1039 and a second extension extending from the biasing member 1030 having an engagement protrusion 1032, e.g., a knob, a notch, an extending tab, a protrusion and the like. The engagement protrusion 1032 is sized and configured to reside at least partially inside a notch 1038 to secure the attachment member 1008. The first adjustable attachment mechanism 1034 can rotate about biasing member 1030 with application of force to the tab 1039 in the direction of arrow 1035. Upon applying force to tab 1039 the first adjustable attachment mechanism 1034 moves to release the engagement protrusion 1032 from a notch 1038. The first adjustable attachment mechanism 1034 and second adjustable attachment mechanism 1036 can constructed from plastic, thermoplastic, polymer, combinations of the same and the like. In a preferred embodiment, the first adjustable attachment mechanism 1034 and the second adjustable attachment mechanism 1036 are constructed from Acetal plastic.

In addition, mounted on an interior surface of the front side upper backplate 1005 is second adjustable attachment mechanism 1036. The second adjustable attachment mechanism 1036 includes a biasing member 1040, e.g., a spring or other biasing means, attached to front side the upper back plate 1005, a first extension extending from the biasing member 1040 to a tab 1042 and a second extensions extending from the biasing member 1040 to an engagement protrusion 1044, e.g., a knob, a notch, an extending tab, a protrusion, and the like. The engagement protrusion 1044 is sized and configured to reside at least partially inside a notch 1038 to secure the attachment member 1008. The second adjustable attachment mechanism 1036 can rotate about biasing member with application of force to the tab 1042 and rotate in the direction of arrow 1037. Upon applying a force to tab 1036 the second adjustable attachment mechanism 1036 moves to release the engagement protrusion 1040 from a notch 1038. In a resting configuration the biasing members 1040, 1030 apply force to ensure the engagement protrusions 1044, 1032 are under force to remain in their respective locked configurations in respective notches 1038.

In operation, a force is applied to the tabs 1036, 1042 to release the engagement protrusion 1044 and 1032 from the notches 1038, thereby allowing the attachment member 1008 to move up and down to a desired location. Releasing the tabs 1036, 1042 allows the engagement protrusions 1044, 1032, to engage with notches 1038, thereby locking the attachment member 1008 at the desired portion with the biasing members described herein. In a preferred embodiment, the force is applied substantially simultaneously to each tab 1039, 1042, e.g., by pinching tabs 1039 and 1049 at substantially the same time, thereby releasing the engagement protrusions 1044, 1032, substantially simultaneously.

Again referring to FIG. 10E, a disassembled view of the upper backplate 1006 showing a front side upper back plate 1005 with the back side upper back plate 1007 removed is shown. A first channel 1020 and a second channel 1022 are formed between the front side upper backplate 1005 and the backside upper backplate 1007. The channel 1020 is sized and configured to receive a portion of the first shoulder extension 1002. The channel 1022 is sized and configured to receive a second shoulder extension 1004. The first shoulder extension 1002 and second shoulder extension 1004 are attached to the front side upper back plate 1005 and back side upper backplate 1007 with one or more attachment mechanisms through holes 1024, e.g., screw, pin, rivet, combination of the same and the like. In a preferred embodiment, coated metal, alloy screws, oxide-coated metal, e.g., black oxide-coated steel, such as are utilized to attach the first shoulder extension 1002 and the second shoulder extension 1004. In addition, the front side upper back plate 1005 is coupled to the back side upper backside 1007 with one or more attachment mechanisms through one or more holes 1013. The attachment mechanisms are described herein, e.g., screws, alloy screws, rivets, pegs, etc. In a preferred embodiment, anodized steel is used.

Referring to FIG. 10F, a lower backplate 1012 includes a front side lower backplate 1057 and a back side lower backplate 1054. Referring to FIG. 10G, the back side lower backplate 1054 has been removed. Attachment member 1008 includes a plurality of side notches 1038 spaced at predetermined intervals to aid with adjusting the distance between the upper back plate 1006 and the lower back plate 1012. Mounted on an interior surface of the front side lower back plate 1054 is a first adjustable attachment mechanism 1051 and a second adjustable attachment mechanism 1063. The first adjustable attachment mechanism 1051 includes a biasing member 1058, e.g., a spring or other biasing means, attached to the lower back plate 1012, a first extension extending from the biasing member 1058 with a tab 1060 and a second extension extending from the biasing member 1058 having an engagement protrusion 1053, e.g., a knob, notch, extending tab, protrusion, and the like. The engagement protrusion 1053 is sized and configured to reside at least partially inside a notch 1038 to secure the attachment member 1008. The first adjustable attachment mechanism 1051 can rotate about the biasing member 1058 with application of force to the tab 1060 and the rotation is in a direction 1061. Upon applying force to the tab 1060 the first adjustable attachment mechanism 1051 moves to release the engagement protrusion 1053 from the notch 1038.

In addition, mounted on an interior surface of the lower plate 1012 is second adjustable attachment mechanism 1063. The second adjustable attachment mechanism 1063 includes a biasing member 1066, e.g., a spring or other biasing means, attached to the lower backplate 1012, a first extension extending from the biasing member 1066 with a tab 1068 and a second extension extending from the biasing member 1063 having an engagement protrusion 1064, e.g., a knob, notch, extending tab, protrusion, and the like. The engagement protrusion 1064 is sized and configured to reside at least partially inside a notch 1038 to secure the attachment member 1008. The second adjustable attachment mechanism 1063 can rotate about the biasing member 1066 with application of force to the tab 1042 and rotate in a direction 1070. Upon applying a force to the tab 1068 the second adjustable attachment mechanism 1063 moves to release the engagement protrusion 1064 from the notch 1038. In a resting configuration the biasing members 1063, 1051 apply force to ensure the engagement protrusions 1053, 1064 are under force to remain in their respective locked configurations in respective notches 1038.

In operation, a force is applied to the tabs 1068, 1060 to release the engagement protrusions 1053, 1064 from the notches 1038, thereby allowing the attachment member 1008 to move up and down to a desired location. In a preferred embodiment, the force is applied substantially simultaneously, e.g., by pinching tabs 1060 and 1068 at substantially the same time, thereby releasing the engagement protrusions 1064, 1053 substantially simultaneously. The

FIG. 10L is a right side front perspective view of FIG. 10A with a waist belt and padding according to another embodiment of the invention;

Referring to FIG. 10L, the weight transfer support device 1000 includes a first shoulder extension 1002 and a spaced apart second shoulder extensions 1004, each extending upward and away from an upper backplate 1006. The upper backplate 1006 is adjustably connected to lower backplate 1012 with an attachment member 1008. In this embodiment, the lower backplate 1012 includes a waist belt 1082 connected to the lower backplate 1012 through an opening 1086 and attachment mechanism, e.g., pull cord, snaps, magnets, screws, combinations of the same and the like. That is, the lower back plate 1012 is received within an opening 1086. The waist belt 1082 is configured to couple to the waist of a user and has a first extension 1088 and a second extension 1090 and an attachment mechanism 1096. The attachment mechanism 1096 is a buckle but could be any attachment mechanism configured to releasably attach the first extension 1088 and the second extension 1090. The waist belt 1082 can have an inner belt as described herein. In addition, the waist belt 1082 can have attachment points for the radiation shielding apparatus as described herein. The waist belt can also have padding for comfort of the user.

The upper plate has a padding 1080 releasably coupled to the upper backplate 1006. In this embodiment there are openings to receive the shoulder extensions 1004, 1002, upper plate 1006 and attachment member 1008. Optionally and/or alternatively, the padding can be adhered to a surface of the upper back plate 1006. The padding can be a foam padding, a quilted padding, coating, or other similar padding.

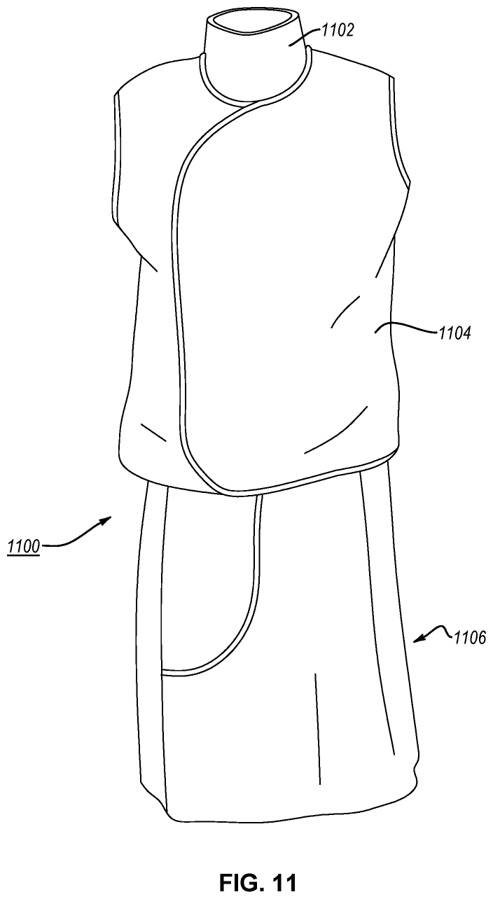

FIG. 11 is front view of a radiation shielding apparatus according to an embodiment of the invention.

Referring to FIG. 11, the radiation shielding apparatus is generally depicted with reference to 1100. The radiation shielding apparatus 1100 includes a radiation shielding scarf or thyroid shield 1102, a radiation shielding vest 1104, and a radiation shielding skirt 1106. In one embodiment, the radiation shielding vest 1104 is sized to have length about the same size as the skirt 1106, thereby eliminating the need for the skirt 1106.

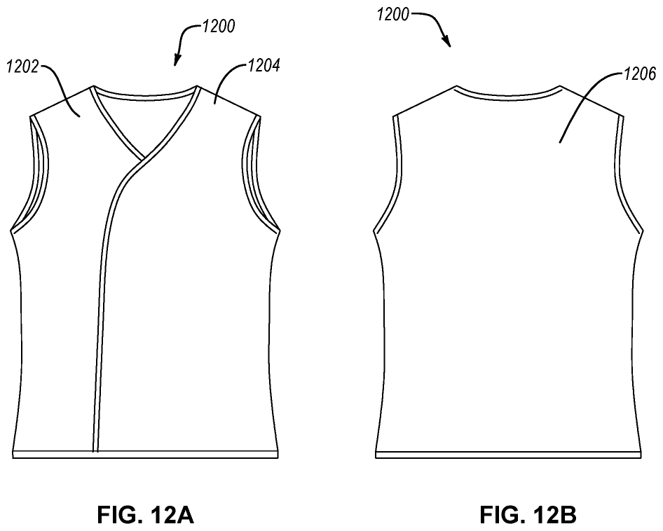

FIG. 12A is a front view of a radiation shielding vest according to an embodiment of the invention. FIG. 12B is a back view of a radiation shielding vest according to an embodiment of the invention.

Referring to FIGS. 12A-12B, a radiation shielding vest or an upper portion of a radiation shielding apparatus is shown with reference to 1200. The radiation shielding vest 1200 includes a first right side 1202, a second left side portion 1204, and a back portion 1206. The right side portion 1202 is overlayed by the left side portion 1204.

As described herein, the radiation shielding apparatus 1200 is configured to be worn by a user, such as health professionals, to attenuate radiation in cath labs, radiology rooms, and health imaging departments. Common industry standards and requirements for lead-based radiation shielding systems include 0.25 and 0.5 mm “lead equivalence.” In one exemplary embodiment, the core material includes 0.25 mm lead equivalence. However, it is understood that the core material may include anywhere between 0.25 mm to 0.5 mm lead equivalence.

The first right side portion 1202 may be secured to the second left side portion 1204 by a buckle, belt, hook-and-loop fastener, combinations of the same and the like. The first right side 1202 is overlayed by the left side portion 1204 to ensure sufficient torso coverage from radiation. Since health professionals and other users are generally front-facing a radiation source, the front right side portion 1202 and second left side portion 1204 may have as thick or thicker lead core material as necessary for each desired radiation exposure application. The vest can include a dosimeter or use meter as described herein. In addition, the vest 1200 can also include one or more external or internal pockets to aid the user with storage of items.

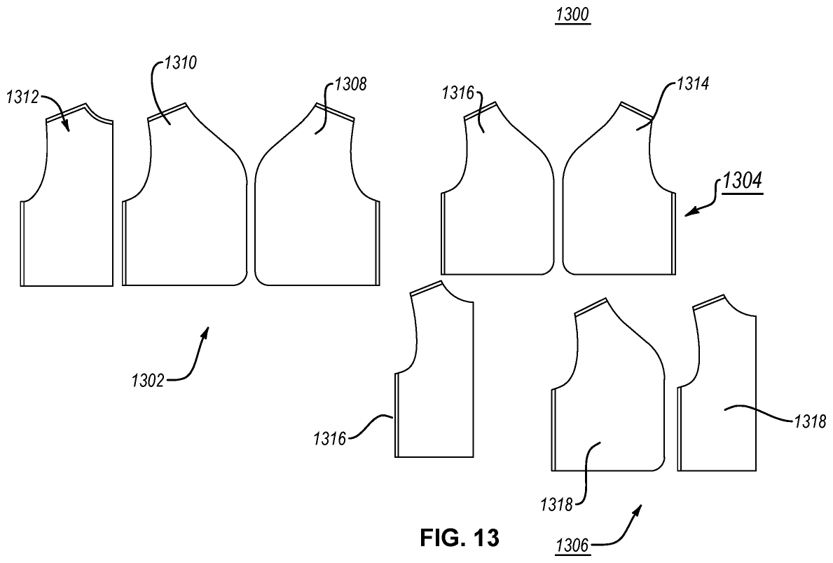

FIG. 13 is a dissembled view of the radiation shielding vest of FIGS. 11A and 11B according to an embodiment of the invention.

Referring to FIG. 13, the radiation shielding apparatus 1300 includes outer layers 1302, inner layers 1304, and radiation shielding layers or core material layers 1306 for a radiation shielding vest.

The outer layers 1302 includes a right outer layer 1308, a left outer layer 1310, a back outer layer 1312 in a folded layout. The inner layer 1304 includes a right inner layer 1314, a left inner layer 1316, a back inner layer 1316 similar to 1312 in a folded layout. The radiation shielding layer or core material layer 1306 includes a right core layer 1318, a left right core layer (not shown) similar to 1310, a back core layer 1319 in a folded layout.

The left outer layer 1310 and right outer layer 1308 represent an exterior of the apparatus 1300, e.g., radiation-facing layer. The outer layers 1302 and inner layers 1304 may be of any materials described herein, e.g., vinyl, nylon, polyester, polypropylene, rubber, cotton, an elastane mixture, a cotton-polyester, combinations, of the same and the like.

The outer layers 1302 and inner layers 1304 are separated by one or more radiation shielding core materials sandwiched between the inner layers 1304 and outer layers 1302. The core material includes any radiation shielding material described herein, e.g., lead material, non-lead material, light-lead material, combinations of the same and the like. Other high-density materials that exhibit high attenuation of radiation such as, heavy metals tin, antimony, tungsten, and bismuth and titanium amalgamations, lead-polyethylene-boron composites, and the like, may be utilized. In one exemplary embodiment, the core material includes 0.25 mm lead equivalence. The outer layers 1302, inner layers 1304, and radiation shielding layers or core material layers 1306 are constructed as described herein.

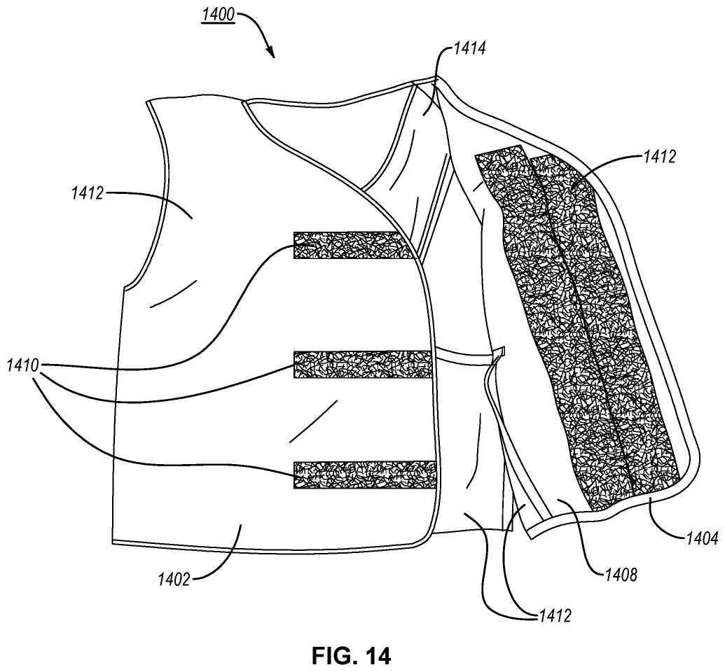

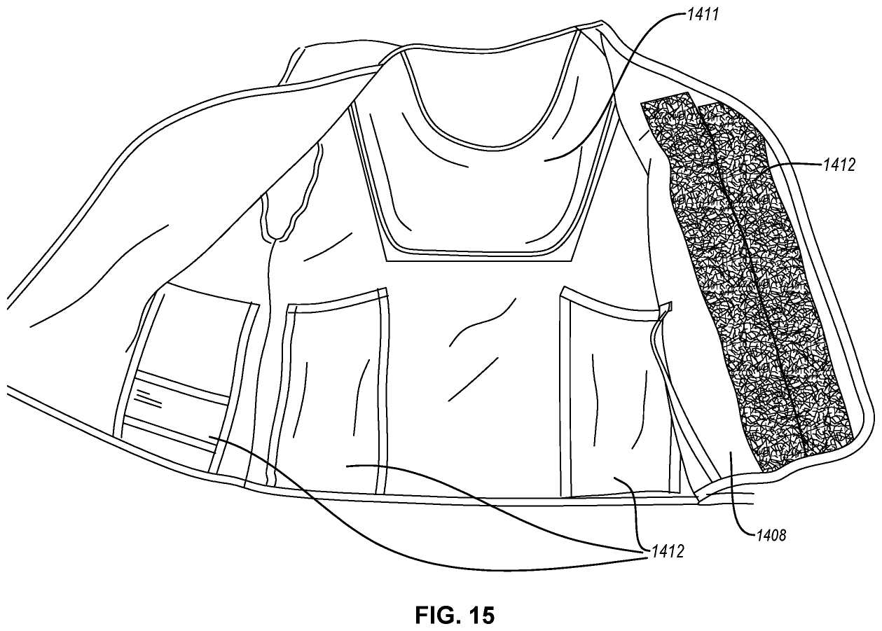

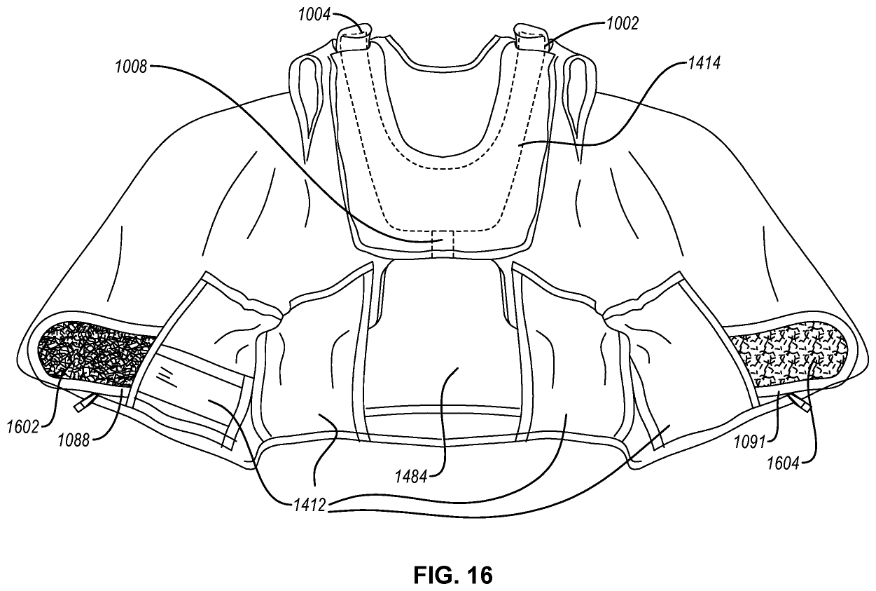

FIG. 14 is an illustrative view of the radiation shielding apparatus in a first orientation according to another embodiment of the invention. FIG. 15 is an illustrative view of the radiation shielding apparatus in a second orientation. FIG. 16 is an illustrative view of FIG. 14 in a third orientation.

Referring to FIGS. 14-16, the radiation shielding apparatus is generally depicted with reference to number 1400. The apparatus 1400 includes a right side 1402 and left side 1404. The right side 1402 has a plurality of hook and loop fasters 1410, the left side 1404 has an inner side 1406 including a plurality of hook-and-loop fasteners 1408 configured to enable the right side 1402 to be releasably attached to the left side 1404. The vest 1400 may further include a plurality of interior attachment mechanisms 1412, e.g., tube portions, or loop portions such as belt loops configured to couple the vest 1400 to corresponding portions of a weight supporting device and/or waist strap of weight supporting device. The vest 1400 may further include an interior shoulder extension attachment mechanism 1414, e.g., tube portion, loop portions such as belt loops configured to couple to each shoulder extension or other location of the weight supporting device described herein. In one embodiment, the waist belt extensions 1088 and 1090 are arranged through the attachment mechanisms 1412 and include hook and loop 1602, 1604, fasteners that can be connected together. The shoulder extension members 1004, 1002 are arranged in attachment mechanisms 1414.

Optionally and/or alternatively, the inside of the vest includes hook and loop attachment mechanisms configured to correspond to hook and loop attachment mechanisms on a waist belt 1082. In one embodiment, the attachment mechanisms, e.g., hook and loop, on the waist belt are on a back side of the belt 1084 and/or extensions 1088 and 1082 and those attachment mechanisms correspond to attachment mechanisms, e.g., hook and loop mechanisms, on the vest 1400, e.g., an internal surface.

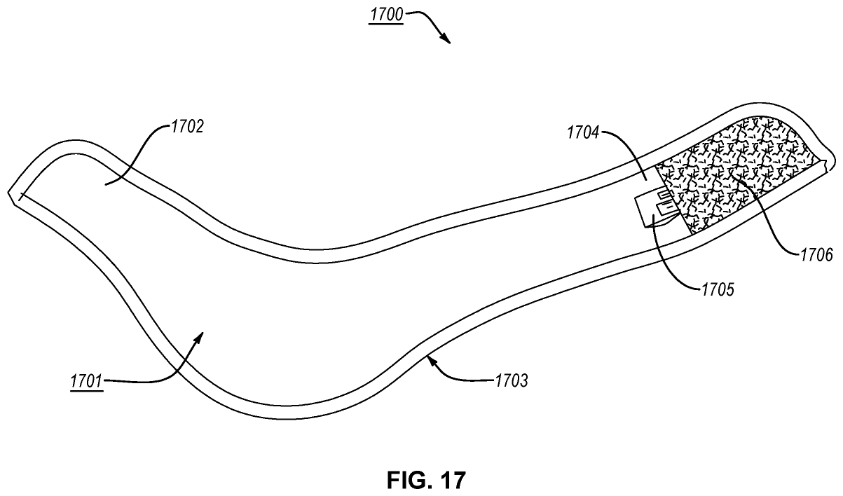

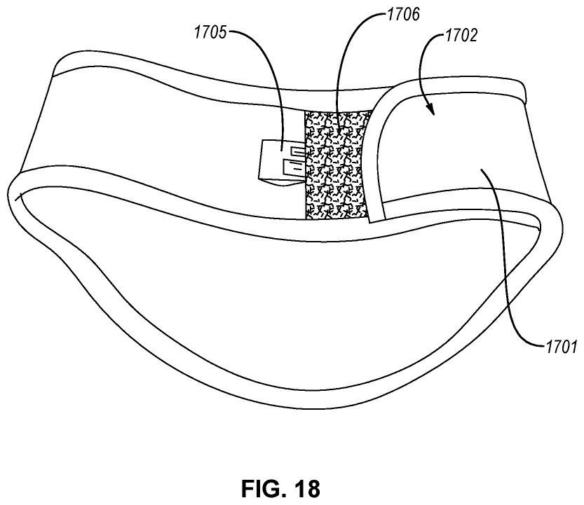

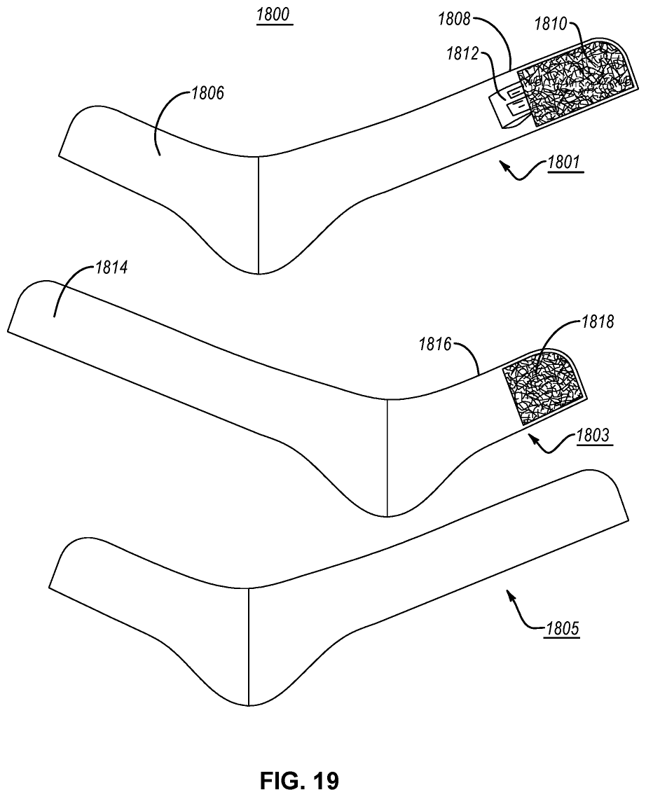

FIG. 17 depicts a radiation shielding apparatus in a first orientation according to another embodiment of the invention. FIG. 18 depicts a radiation shielding apparatus of FIG. 17 in a second orientation according to another embodiment of the invention. FIG. 19 depicts a radiation shielding apparatus of FIG. 17 in a disassembled view.

Referring to FIGS. 17-18, the radiation shielding apparatus 1700 includes a scarf or thyroid shield. The scarf 1700 includes a first end portion 1702 and opposing second end portion 1704. The scarf 1700 includes an inside surface 1703 and outside surface 1701. The scarf includes a tag 1705 including a dosimeter and/or usage meter. The usage meter can be a manual usage meter configured to indicate the number of times the garment is exposed to radiation. The first end portion 1702 inside surface 1703 includes a hook and loop region and the second end portion 1704 outside surface 1701 includes a hook and loop region 1706. The hook and loop regions are configured to releasably couple the first end region 1702 to the second end region 1704 such that the scarf 1700 can encircle a user's neck, covering the user's neck and protecting the neck region from radiation. The scarf 1700 includes an inner layer, an outer layer and radiation shielding layer or core layer as described herein.

Referring to FIG. 19, again the apparatus 1800 is in the form of a scarf configured to shield a user's thyroid and neck region in a disabled view. The scarf 1800 includes an inner layer 1803, an outer layer 1801, and radiation shielding layer or core region 1805.

The outer layer 1801 includes a first end region 1806, an opposite second end region 1808. The second end region 1808 includes a hook-and-loop fastener 1810 and a tag 1812. The tag can include a dosimeter and/or usage meter as described herein. The inner layer 1803 includes a first end region 1814 and an opposite second end region 1816. The second end region 1816 includes a hook-and-loop fastener 1818 configured to releasably attach to the hook and loop fastener 1810.

The radiation shielding layer 1805 includes a radiation shielding material as described herein, e.g., lead, light lead, and/or non-lead material. In one embodiment, the radiation shielding material includes, e.g., high-density materials that exhibit high attenuation of radiation, such as, heavy metals tin, antimony, tungsten, and bismuth and titanium amalgamations, lead-polyethylene-boron composites, and the like. In one exemplary embodiment, the core material includes 0.25 mm lead equivalence. The outer layer 1801 and inner layer 1803 include a material described herein, e.g., such as vinyl, nylon, polyester, polypropylene, rubber, cotton, an elastane mixture, a cotton-polyester combination, and/or the like.

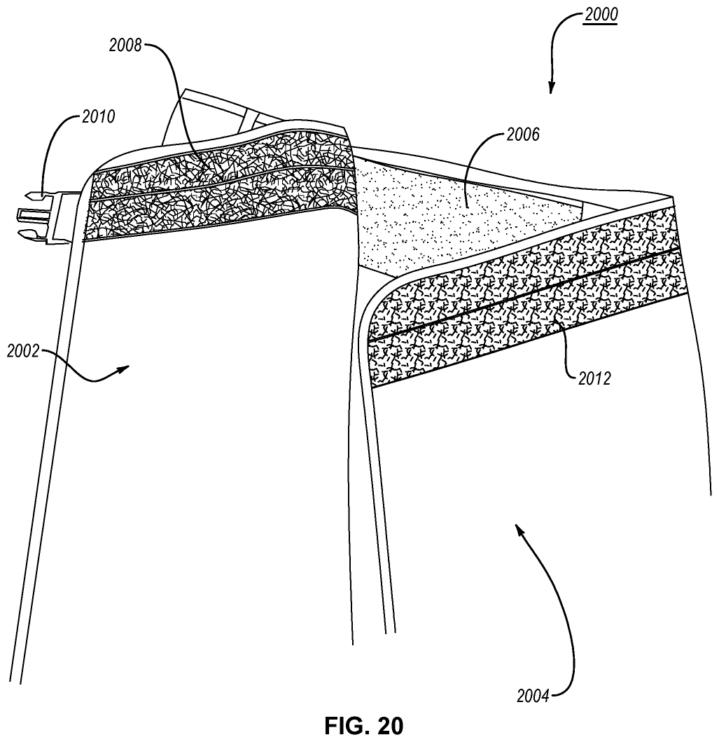

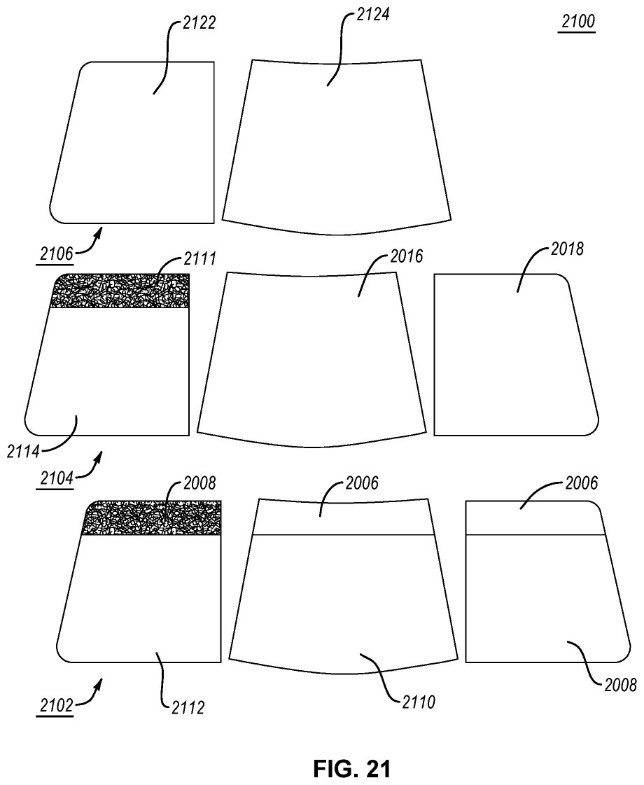

FIG. 20 depicts a radiation shielding apparatus in a first orientation according to another embodiment of the invention. FIG. 21 depicts a radiation shielding apparatus of Figure in a disassembled view.

Referring to FIG. 20, the radiation shielding apparatus is generally depicted as reference number 2000. The radiation shielding apparatus 2000 is in the form of a skirt. It includes an inside surface 2002 and an outside surface 2004. The radiation shielding apparatus 2000 includes a top portion configured to go around a user's waist. The inside surface 2002 includes a grip region 2006 at least partially around top portion that corresponds at least partially to a waist of a user, and a hook and loop region 2008. The outside surface 2004 includes a hook and loop region 2012 configured to be releasably coupled to the hook and loop region 2008. Of course, other attachment mechanisms can include snaps, buttons, magnets, combinations of the same and like. A buckle 2010 is configured to attach to an opposing buckle (not shown) with an adjustable belt strap around a top portion of the skirt.

FIG. 21 is a disassembled view of a radiation shielding apparatus 2100. Referring to FIG. 21, the radiation shielding apparatus 2100 is in the form of a skirt configured to shield a user's lower body region. The skirt 2100 includes an inner layer 2102, an outer layer 2104, and radiation layer or core region 2106. The inner layer 2102 includes a first side panel 2112, a center panel 2110, and a second side panel 2108. The top portion of the center panel 2110 and the second side panel 2108 includes a grip material 2006. The grip material 2006 includes a friction coating or layer, e.g., Mega Grip material from TVF Inc. The top region of the first side panel 2112 includes a hook-and-loop fastener 2008.

The outer layer 2104 includes a first side panel 2114, a center panel 2116, a second side panel 2118 and a hook and loop region 2111. The first side panel 2114 includes a hook-and-loop fastener 2111 configured to releasably attach to the hook and loop fastener 2008.

An inner radiation shielding layer 2106 includes a radiation shielding material as a first panel 2124 and second panel 2122, e.g., lead, light lead, and/or non-lead material. In one embodiment, the radiation shielding material includes, e.g., high-density materials that exhibit high attenuation of radiation, such as, heavy metals tin, antimony, tungsten, and bismuth and titanium amalgamations, lead-polyethylene-boron composites, and the like. In one exemplary embodiment, the core material includes 0.25 mm lead equivalence. The outer layer 2102 and inner layer 2104 include a material described herein, e.g., such as vinyl, nylon, polyester, polypropylene, rubber, cotton, an elastane mixture, a cotton-polyester combination, and/or the like. The outer layer 2102, inner layer 2104 and radiation layer 2106 are joined together as described herein.

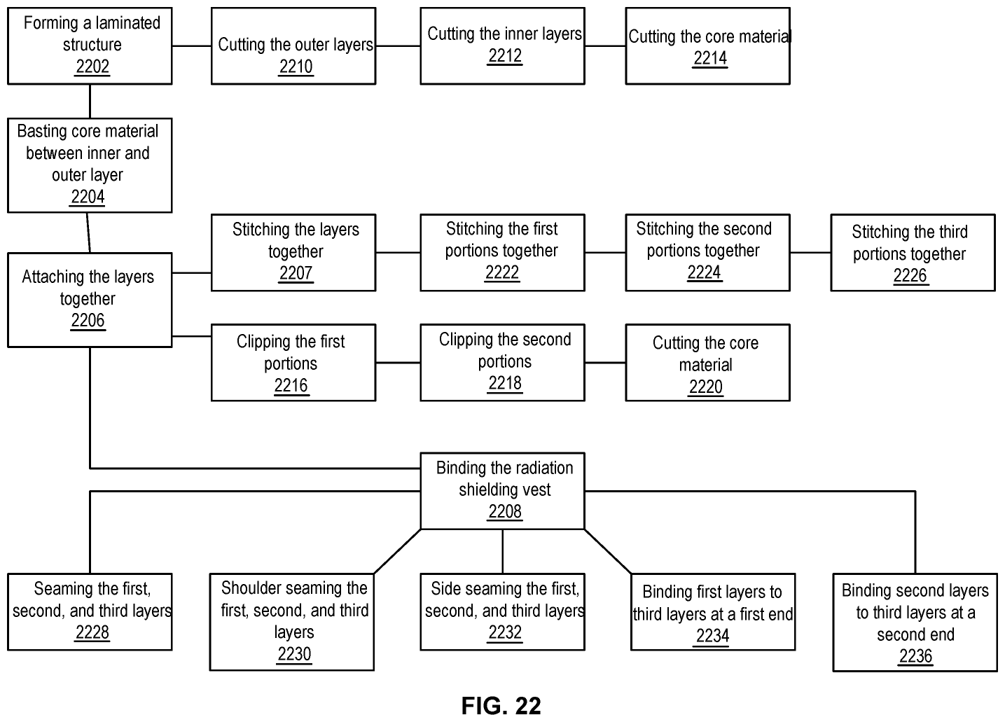

FIG. 22 illustrates a method of making a radiation apparatus according to an embodiment of the invention.

Referring to FIG. 22, the method of making a radiation apparatus is generally depicted with reference to number 2200. The method 2200 includes arranging a laminated structure (step 2202), e.g., having an outer layer, an inner layer, and a core material, configured to be arranged between the outer and inner layer. The method may further include basting (step 2204) the core material between the outer layer and the inner layer, wherein the basting (step 2204) includes attaching (step 2206) (e.g., clipping) the layers together, e.g., by stitching (step 2207) the layers together or connecting them using other suitable techniques, e.g., fastening, zipping, adhering, fusing, tacking, seaming, hemming, combinations of the same and the like. In various embodiments, the method 2200 includes binding (step 2208) the radiation shielding vest to form a wearable one-piece garment configured to cover a user's torso. In one embodiment, the outer layer includes vinyl, nylon, polyester, polypropylene, rubber, cotton, an elastane mixture, a cotton-polyester combination, and/or the like.

In one embodiment, the inner layer includes materials described herein, e.g., vinyl, nylon, polyester, polypropylene, rubber, cotton, an elastane mixture, a cotton-polyester combination, and/or the like. In one embodiment, the core material is a radiation shielding material as described herein, e.g., a material configured to attenuate, i.e., block, radiation. The core material can include lead, non-lead, and light materials as described herein. In one embodiment, the radiation shielding material includes one or more of heavy metals tin, antimony, tungsten, and bismuth and titanium amalgamations, lead-polyethylene-boron composites, and the like. In one exemplary embodiment, the core material includes 0.25 mm lead equivalence.

In one embodiment, the forming (step 2202) the laminate outer layer includes cutting (step 2210) a first outer layer, a second outer layer, and a third outer layer. In various embodiments, the forming (step 2202) the laminate inner layer comprises cutting (step 2212) a first inner layer, a second inner layer, and a third inner layer, wherein the third inner layer comprises a plurality of snap settings. The plurality of snap settings may be configured to connect the inner layer to a weight transfer device (e.g., exoskeleton). In various embodiments, the forming (step 702) the laminate core material comprises cutting (step 2214) at least two first core materials, at least two second core materials, and at least one third core material.

In one embodiment, the attaching (step 2206) (e.g., clipping) includes clipping (step 2216) the first outer layer, the first inner layer, and the at least two first core materials together. In various embodiments, the attaching (step 2206) may comprise clipping (step 2218) the second outer layer, the second inner layer, and the at least two second core materials together. In various embodiments, the attaching (step 2206) may also include clipping (step 2220) the third outer layer, the third inner layer, and the at least one third core material together.

In one embodiment, the stitching (step 2207) includes stitching (step 2222) the first outer layer, the first inner layer, and the at least two first core materials together. In various embodiments, the stitching (step 2207) may comprise stitching (step 2224) the second outer layer, the second inner layer, and the at least two second core materials together. In various embodiments, the stitching (step 2207) may comprise stitching (step 2226) the third outer layer, the third inner layer, and the at least one third core material together. The core materials may include lead and be configured to attenuate, e.g., block, radiation waves. Other high-density materials that exhibit high attenuation of radiation, such as heavy metals tin, antimony, tungsten, and bismuth and titanium amalgamations, lead-polyethylene-boron composites, and the like, may be utilized. In one exemplary embodiment, the core material includes 0.25 mm lead equivalence.

In one embodiment, the binding (step 2208) includes seaming (step 2228) each of the first layers, second layers, and third layers. In various embodiments, the binding (step 2208) can also include shoulder seaming (step 730) each of the first layers, second layers, and third layers. In various embodiments, the binding (step 708) may comprise side seaming (step 2232) each of the first layers, second layers, and third layers. In various embodiments, the binding (step 2208) may comprise binding (step 2234) the first layers to the third layers at a first end. In various embodiments, the binding (step 2208) may comprise binding (step 2236) the second layers to the third layers at a second end.

Particular example embodiments of the subject matter have been described. As will be apparent to those skilled in the art, other embodiments, implementations, alterations, and permutations of the particular implementations are considered to be within the scope of the disclosure and the following claims. Features of the various embodiments are also combinable. While operations are depicted in the drawings or claims in a particular order, this should not be understood as requiring that such operations be performed in the particular order shown or in sequential order, or that all illustrated operations be performed (some operations may be considered optional), to achieve desirable results.

While this disclosure contains many specific embodiments details, these should not be construed as limitations on the scope of any invention or on the scope of what may be claimed, but rather as descriptions of features that may be specific to particular embodiments of particular inventions. Certain features that are described in the context of separate embodiments can also be implemented, in combination, in a single embodiment.

Conversely, various features that are described in the context of a single embodiment can also be implemented in multiple embodiments separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Accordingly, the previously described example embodiments do not necessarily define or constrain this disclosure. Other changes, substitutions, and alterations are also possible within the scope of this disclosure.

To avoid unnecessarily obscuring the present disclosure, the preceding description may omit a number of known structures and devices. This omission is not to be construed as a limitation of the scopes of the claims. Specific details are set forth to provide an understanding of the present disclosure. It should however be appreciated that the present disclosure may be practiced in a variety of ways beyond the specific detail set forth herein.

Also, while the flowcharts have been discussed and illustrated in relation to a particular sequence of events, it should be appreciated that changes, additions, and omissions to this sequence can occur without materially affecting the operation of the disclosed embodiments, configuration, and aspects. A number of variations and modifications of the disclosure can be used. It would be possible to provide for some features of the disclosure without providing others.

Moreover, though the description has included a description of one or more aspects, implementations, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A radiation shielding system, comprising:
a weight supporting device, wherein the weight supporting device includes an exoskeleton configured to bear a weight of a radiation shielding garment, wherein the radiation shielding garment is configured to be arranged and at least partially supported by the weight supporting device, wherein the radiation shielding garment comprising:
a vest;
a skirt; and
a scarf,
wherein the vest and the skirt are configured to be at least partially supported by the weight supporting device.

2. The system of claim 1, wherein the scarf comprises opposing ends configured to be at least partially coupled by a hook-and-loop fastener.

3. The system of claim 1, wherein the skirt comprises opposing ends configured to be at least partially coupled by a buckle.

4. The system of claim 1, wherein the vest comprises opposing ends configured to be at least partially coupled by a buckle and a plurality of hook-and-loop fasteners.

5. The system of claim 1, wherein each of the vest, skirt, and scarf comprise a laminate having an outer layer, an inner layer, and at least one core material between the outer layer and inner layer.

6. The system of claim 5, wherein the core material is lead.

7. A method of assembling a radiation shielding vest, comprising:
forming a laminate having an outer layer, an inner layer, and a core material configured to be arranged between the outer and inner layer;
basting the core material between the outer layer and the inner layer, wherein the basting comprises:
clipping the layers together;
stitching the layers together; and
binding the radiation shielding vest.

8. The method of claim 7, wherein forming the laminate outer layer comprises cutting a first outer layer, a second outer layer, and a third outer layer.

9. The method of claim 8, wherein forming the laminate inner layer comprises cutting a first inner layer, a second inner layer, and a third inner layer, wherein the third inner layer comprises a plurality of snap settings.

10. The method of claim 9, wherein forming the laminate core material comprises cutting at least two first core materials, at least two second core materials, and at least one third core material.

11. The method of claim 10, wherein the clipping comprises:
clipping the first outer layer, the first inner layer, and the at least two first core materials together;
clipping the second outer layer, the second inner layer, and the at least two second core materials together; and
clipping the third outer layer, the third inner layer, and the at least one third core material together.

12. The method of claim 11, wherein the stitching comprises:
stitching the first outer layer, the first inner layer, and the at least two first core materials together;
stitching the second outer layer, the second inner layer, and the at least two second core materials together; and
stitching the third outer layer, the third inner layer, and the at least one third core material together.

13. The method of claim 12, wherein the binding comprises:
an edge seaming each of the first layers, second layers, and third layers;
a shoulder seaming each of the first layers, second layers, and third layers;
a side seaming each of the first layers, second layers, and third layers;
a binding the first layers to the third layers at a first end; and
a binding the second layers to the third layers at a second end.

14. The system of claim 6, further comprising an active cooling means.

15. The system of claim 14, wherein the active cooling means is a heat exchanger.

16. The system of claim 14, wherein the active cooling means is a forced convectional cooling fan.

17. The system of claim 6, further comprising a passive cooling means.

18. The system of claim 6, further comprising a dosimeter configured to monitor radiation shielding garment usage.

19. The system of claim 5, wherein the core material comprises one or more of a light lead material, a lead material, and a lead free material configured to a have a lead equivalence in range from about 0.125 mm to about 1.0 mm or greater.

20. The system of claim 5, wherein the core material comprises one or more of layers of radiation shielding material configured to a have a lead equivalence in range from about 0.125 mm to about 1.0 mm.
